(19) United States
(12) Patent Application Publication
(10) Pub. No.: US 2024/0307628 A1
(43) Pub. Date: Sep. 19, 2024

(54) SYSTEMS AND METHODS FOR DELIVERY OF LIQUID PHARMACEUTICAL COMPOSITIONS IN PARTICULAR COMPRISING ONE OR MORE SGLT-2 INHIBITOR(S)

(71) Applicant: Boehringer Ingelheim Vetmedica GmbH, Ingelheim am Rhein (DE)

(72) Inventors: Claudius WEILER, Ingelheim am Rhein (DE); Stefan Johannes LEHNER, Ingelheim am Rhein (DE)

(21) Appl. No.: 18/594,627

(22) Filed: Mar. 4, 2024

(30) Foreign Application Priority Data
Mar. 6, 2023 (EP) 23 160 128.7

(51) Int. Cl. A61M 5/315; A61K 31/7048

(52) U.S. Cl. CPC A61M 5/31513; A61K 31/7048

(57) ABSTRACT

Systems and methods are described for treatment and/or prevention of a metabolic disorder and/or another medical condition in a patient (e.g., a feline) by administering to the patient a liquid pharmaceutical composition, preferably including one or more SGLT-2 inhibitor compound(s), via a delivery system that includes a syringe. The syringe includes features that facilitate easy, safe and effective doses of small volumetric amounts of the liquid pharmaceutical composition to the patient during administration.

### CROSS-REFERENCE TO RELATED APPLICATION

This application claims priority from European Patent Application No. 23 160 128.7, filed on Mar. 6, 2023, the entire disclosure of which is incorporated herein by reference.

### INCORPORATION BY REFERENCE

All references cited herein, are incorporated by reference herein, in their entirety.

### FIELD OF THE INVENTION

The invention relates to the field of medicine, particularly veterinary medicine. In particular, the invention relates to delivery systems including syringes and corresponding methods that deliver liquid pharmaceutical compositions, in particular comprising one or more SGLT-2 inhibitor compound(s), to a patient.

### BACKGROUND OF THE INVENTION

The treatment of metabolic disorders in mammals includes the inhibition of the renal sodium-dependent glucose co-transporter SGLT-2. SGLT-2 in the kidney regulates glucose levels by mediating the reabsorption of glucose back into the plasma following filtration of the blood. SGLT-2 inhibition thus induces glucosuria and may reduce blood glucose levels.

For example, companion animals including feline animals (e.g., cats) and canine animals (e.g., dogs) can be affected by various metabolic disorders, including hyperglycemia, insulin resistance, diabetes (such as diabetes mellitus type 1 or type 2, or pre-diabetes), hepatic lipidosis, obesity, hyperinsulinemia, impaired glucose tolerance, ketosis (in particular ketoacidosis), dyslipidemia, dysadipokinemia, obesity, subclinical inflammation or systemic inflammation, in particular low grade systemic inflammation, which also comprises adipose tissue, Syndrome X (metabolic syndrome), atherosclerosis and/or inflammation of the pancreas. Various correlations exist amongst these disorders. Among these disorders, diabetes and, in particular pre-diabetes and diabetes mellitus type 2, as well as hyperglycemia, insulin resistance, hepatic lipidosis, and obesity, are gaining more and more importance. Administration of pharmaceutical compositions comprising one or more SGLT-2 inhibitors for the treatment and/or prevention of metabolic disorders in feline and canine animals has been described, e.g., in WO 2015/091313 and WO 2015/110402.

Equine animals (e.g., horses) are affected by various metabolic disorders, including insulin resistance and hyperinsulinaemia. Such insulin-related disorders in equine animals, for example, are only rarely associated with diabetes mellitus and hyperglycemia, as it is in humans or various other mammals. However, in equine animals, insulin also regulates vital metabolic functions; e.g., insulin drives glucose into tissues such as liver, adipose, and skeletal muscle; induces vasoconstrictive and vasodilatory pathways; and regulates protein and fat metabolism. Insulin-related disorders thus have a severe and life-threatening impact on the health of equine animals. They are correlated or may be associated with a number of further equine disorders, conditions or syndromes, including impaired glucose tolerance, dyslipidemia, dysadipokinemia, obesity and/or regional adiposity, subclinical inflammation or systemic inflammation, in particular low grade systemic inflammation, which also comprises adipose tissue, Equine Metabolic Syndrome (EMS) and/or Equine Pituitary Pars Intermedia Dysfunction (PPID), also known as equine Cushing's syndrome, which are characterized e.g. by laminitis, vascular dysfunction, hypertension, hepatic lipidosis, hyperadrenocorticism and/or atherosclerosis. Administration of pharmaceutical compositions comprising one or more SGLT-2 inhibitors for the treatment and/or prevention of metabolic disorders in equine animals has been described, e.g., in WO 2014/161836 and WO 2015/150299.

Other uses of and/or medical indications and/or conditions for the treatment and/or prevention by means of SGLT-2 inhibitors in animals comprise for instance the ones disclosed in WO 2020/219645, WO 2021/105152, WO 2021/165177, WO 2023/006718, WO 2023/006745 and WO 2023/006747.

A large variety of SGLT-2 inhibitors are known and described by the art. A liquid pharmaceutical formulation of SGLT-2 inhibitors is particularly useful to ensure appropriate administration of such compounds in a safe and efficacious manner to the patient. However, it can be a challenge to ensure a precise dosage amount, based upon bodyweight of the patient, is administered at any given time. This is particularly the case for smaller (e.g., companion) animals, including felines and canines, in which the dosage amount might be very small based (e.g., 1 mL or less) upon bodyweight of the animal.

### SUMMARY OF THE INVENTION

In accordance with embodiments described herein, a pharmaceutical delivery system comprises a system for administering a liquid pharmaceutical composition to a patient, the system comprising a syringe. The syringe comprises a barrel comprising a hollow elongated member including an open barrel forward end and an open barrel rear end, the hollow elongated member comprising a first portion that extends from the open barrel forward end to a transition section disposed along the hollow elongated member, the first portion having a first cross-sectional dimension, and a second portion that extends from the transition section to the open barrel rear end, the second portion having a second cross-sectional dimension that is greater than the first cross-sectional dimension. The syringe further comprises a plunger comprising an elongated member including a plunger forward end and a plunger rear end. The plunger and the barrel are dimensioned such that a portion of the plunger including the plunger forward end is insertable within the hollow elongated member of the barrel at the open barrel rear end, and the plunger is extendible within the barrel until the plunger forward end engages with an internal surface portion of the hollow elongated member at the barrel forward end. Receipt of liquid pharmaceutical composition within the barrel of the syringe via the open barrel forward end is limited to a volume defined within the first portion of the barrel, wherein the volume is adjustable by adjusting a displacement of the plunger forward end away from the barrel forward end. Further, a surface wall section of the plunger includes indicia comprising a dosage scale that indicates the volume of the liquid pharmaceutical composition received within the first portion of the barrel based upon a corresponding displacement of the plunger forward end away from the barrel forward end.

In accordance with additional embodiments described herein, one or more SGLT-2 inhibitor compound(s) for use in a method of treating a patient with a liquid pharmaceutical composition comprising providing the syringe and the container that includes the liquid pharmaceutical composition, withdrawing a desired dosage amount of the liquid pharmaceutical composition from the container into the first portion of the barrel of the syringe by displacing the plunger forward end a selected distance from the barrel forward end until a selected mark of the plurality of marks for the dosage scale aligns with an exterior surface of the barrel at the barrel rear end, and administering the desired dosage amount of the liquid pharmaceutical composition from the syringe to the patient. A corresponding method of treating a patient with a liquid pharmaceutical composition, as well as the corresponding use of one or more SGLT-2 inhibitor compound(s) for the preparation of a medicament for treating a patient with a liquid pharmaceutical composition, are also intended to be comprised by the present invention.

The above and still further features and advantages of the present invention will become apparent upon consideration of the following detailed description of specific embodiments thereof.

### BRIEF DESCRIPTION OF THE DRAWINGS

FIG. 1 depicts an elevational view of components of a delivery system (10) for administering a liquid pharmaceutical composition according to embodiments described herein.

FIG. 2 depicts a portion of a container (a cap) in partial cross-section for the delivery system of FIG. 1.

FIG. 3A depicts a perspective view of an adaptor for the container of the delivery system of FIG. 1.

FIG. 3B depicts an elevational side view in cross-section of the adaptor of FIG. 3A.

FIG. 4 depicts a view in elevation of the barrel of a syringe for the delivery system of FIG. 1.

FIG. 5 depicts a view in elevation of the plunger of the syringe for the delivery system of FIG. 1.

FIG. 6 depicts a partial cross-sectional view in elevation of the syringe including plunger engaged with the barrel for the delivery system of FIG. 1.

FIG. 7 depicts an example embodiment of a dosage scale provided on a plunger for a syringe of the delivery system of FIG. 1.

Like reference numerals have been used to identify like elements throughout this disclosure.

### DETAILED DESCRIPTION OF THE INVENTION

Before the embodiments of the present invention are described in further details it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” is omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

Systems are described herein that include a liquid pharmaceutical composition and an administration or delivery device comprising a syringe that is capable of delivering small and precise dosages of the liquid pharmaceutical composition to a patient in an effective manner. As described herein, the liquid pharmaceutical composition in particular comprises one or more SGLT-2 inhibitor compound(s) which, when administered to the patient, provide(s) effective prevention and/or treatment of a metabolic disorder and/or any other medical condition of/for the patient. The liquid pharmaceutical composition can be provided in a tamper-proof container as described herein, where the syringe and container cooperate in a manner which effectively facilitates transfer of a precise volume of the liquid pharmaceutical composition from the container to the syringe during use. The system can further be provided as a kit-of-parts, in which container with liquid pharmaceutical composition and syringe are provided together (e.g., combined in a single package structure) for commercial sale/end use. Alternatively, the liquid pharmaceutical composition can be provided separately from the delivery device.

### Pharmaceutical Compositions

Liquid pharmaceutical compositions for use in the systems as described herein preferably include one or more SGLT-2 inhibitor compound(s) suitable for treatment and/or prevention of a metabolic disorder and/or any other medical condition in a patient. For example, a pharmaceutical composition can include a single SGLT-2 inhibitor compound or, alternatively, two or more SGLT-2 inhibitor compounds.

SGLT-2 inhibitors that can be used in the liquid pharmaceutical compositions for systems described herein include, but are not limited to, glucopyranosyl-substituted benzene derivatives, for example as described in WO 01/27128, WO 03/099836, WO 2005/092877, WO 2006/034489, WO 2006/064033, WO 2006/117359, WO 2006/117360, WO 2007/025943, WO 2007/028814, WO 2007/031548, WO 2007/093610, WO 2007/128749, WO 2008/049923, WO 2008/055870, WO 2008/055940, WO 2009/022020 or WO 2009/022008.

Some non-limiting examples of SGLT-2 inhibitor compounds that can be provided in the liquid pharmaceutical compositions as described herein include the following compounds or pharmaceutically acceptable forms thereof:
(1) a glucopyranosyl-substituted benzene derivative represented by the following formula:

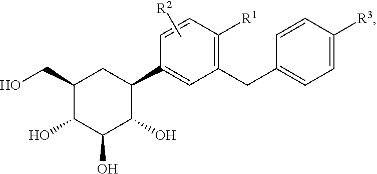

wherein R₁ denotes cyano, Cl or methyl (most preferably cyano),
R₂ denotes H, methyl, methoxy or hydroxy (most preferably H), and
R₃ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R₃ is preferably selected from cyclopropyl, ethyl, ethinyl, ethoxy, (R)-tetrahydrofuran-3-yloxy or (S)-tetrahydrofuran-3-yloxy; and most preferably R₃ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by the following formula:

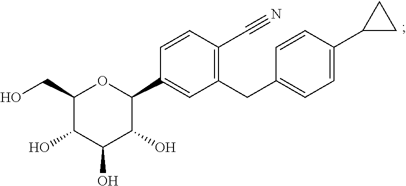 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by the following formula:

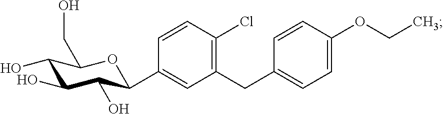

(4) Canagliflozin, represented by the following formula:

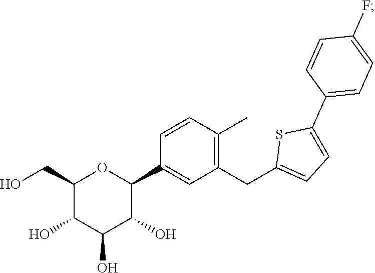 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by the following formula:

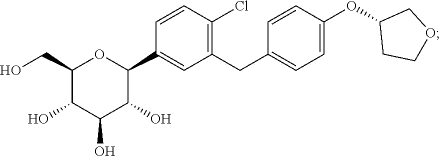 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luscogliflozin, represented by the following formula:

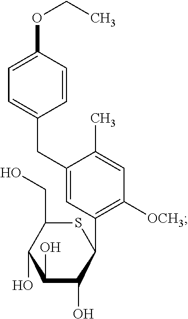

(7) Tofogliflozin, represented by the following formula:

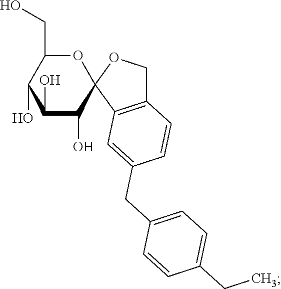

(8) Ipragliflozin, represented by the following formula:

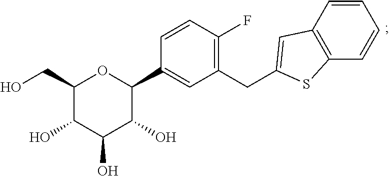 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by the following formula:

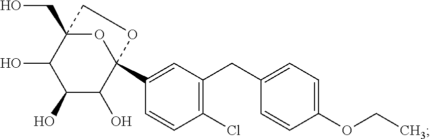 [SMILES: CCOc1ccc(Cc2cc([C@]34OC[C@](CO)(O3)C(O)[C@H](O)C4O)ccc2Cl)cc1]

(10) Atigliflozin, represented by the following formula:

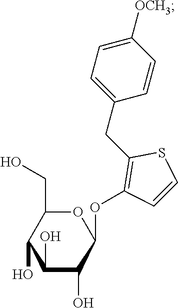

(11) Remogliflozin, represented by the following formula:

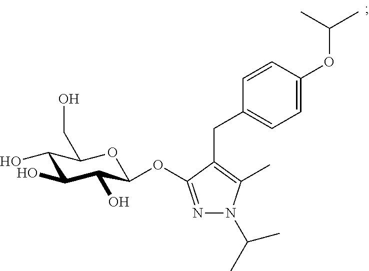

(11A) Remogliflozin etabonate, represented by the following formula:

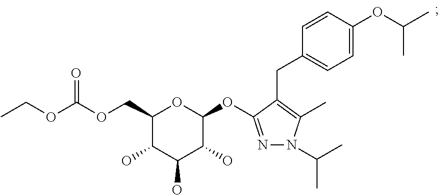 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative represented by the following formula:

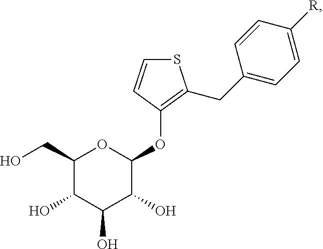

wherein R denotes methoxy or trifluoromethoxy;
(13) 1-(β-D-glucopyranosyl)-4-methyl-3-[5-(4-fluorophenyl)-2-thienylmethyl]benzene, represented by the following formula:

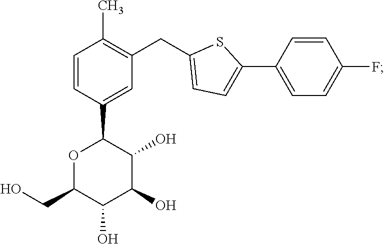 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative represented by the following formula:

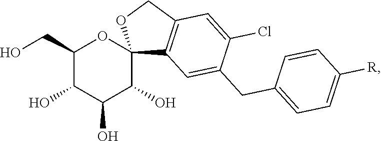

wherein R denotes methoxy, trifluoromethoxy, ethoxy, ethyl, isopropyl or tert. butyl;
(15) a pyrazole-O-glucoside derivative represented by the following formula:

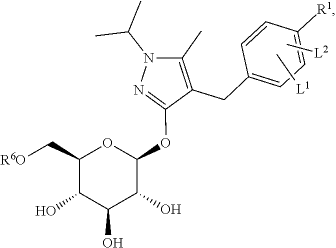

wherein:
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by the following formula:

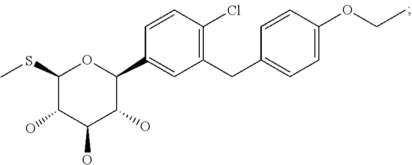 [SMILES: CCOc1ccc(Cc2cc([C@@H]3O[C@H](SC)[C@@H](O)[C@H](O)[C@H]3O)ccc2Cl)cc1]

(17) Sergliflozin, represented by the following formula:

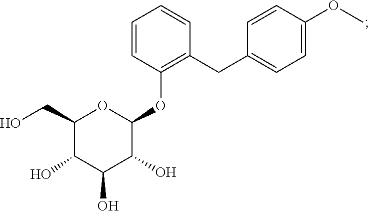 [SMILES: COc1ccc(Cc2ccccc2O[C@@H]2O[C@H](CO)[C@@H](O)[C@H](O)[C@H]2O)cc1]

(18) a compound represented by the following formula:

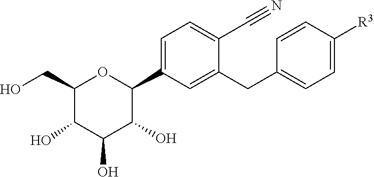

wherein:
R₃ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R₃ is preferably selected from cyclopropyl, ethyl, ethinyl, ethoxy, (R)-tetrahydrofuran-3-yloxy or (S)-tetrahydrofuran-3-yloxy; and R₃ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by the following formula:

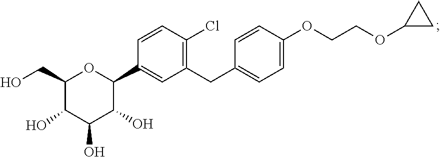 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by the following formula:

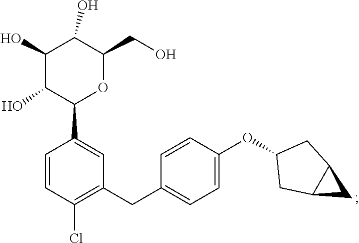 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin;

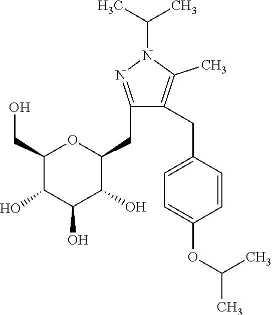 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
(23) Enavogliflozin, represented by the following formula:

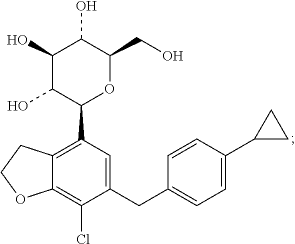 [SMILES: OC[C@H]1O[C@@H](c2cc(Cc3ccc(C4CC4)cc3)c(Cl)c3c2CCO3)[C@H](O)[C@@H](O)[C@@H]1O]

and
(24) TFC-039, represented by the following formula:

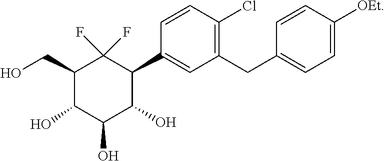 [SMILES: CCOc1ccc(Cc2cc([C@H]3[C@H](O)[C@@H](O)[C@H](O)[C@@H](CO)C3(F)F)ccc2Cl)cc1]

The term “velagliflozin” as employed herein refers to velagliflozin of the above structure as well as pharmaceutically acceptable forms thereof, including hydrates and solvates thereof, and crystalline forms thereof. The compound, methods of its synthesis and co-crystals thereof are described in WO 2007/128749, WO 2014/016381 and WO 2019/121509 for example.

The term “dapagliflozin” as employed herein refers to dapagliflozin of the above structure as well as pharmaceutically acceptable forms thereof, including hydrates and solvates thereof, and crystalline forms thereof. The compound and methods of its synthesis are described in WO 03/099836 for example. Preferred hydrates, solvates and crystalline forms are described in the patent applications WO 2008/116179 and WO 2008/002824 for example.

The term “canagliflozin” as employed herein refers to canagliflozin of the above structure as well as pharmaceutically acceptable forms thereof, including hydrates and solvates thereof, and crystalline forms thereof. The compound and methods of its synthesis are described in WO 2005/012326 and WO 2009/035969 for example. Preferred hydrates, solvates and crystalline forms are described in the patent application WO 2008/069327 for example.

The term “empagliflozin” as employed herein refers to empagliflozin of the above structure as well as pharmaceutically acceptable forms thereof, including hydrates and solvates thereof, and crystalline forms thereof. The compound and methods of its synthesis are described in WO 2005/092877, WO 2006/120208 and WO 2011/039108 for example. A preferred crystalline form is described in the patent applications WO 2006/117359 and WO 2011/039107 for example.

The term “atigliflozin” as employed herein refers to atigliflozin of the above structure as well as pharmaceutically acceptable forms thereof, including hydrates and solvates thereof, and crystalline forms thereof. The compound and methods of its synthesis are described in WO 2004/007517 for example.

The term “ipragliflozin” as employed herein refers to ipragliflozin of the above structure as well as pharmaceutically acceptable forms thereof, including hydrates and solvates thereof, and crystalline forms thereof. The compound and methods of its synthesis are described in WO 2004/080990, WO 2005/012326 and WO 2007/114475 for example.

The term “tofogliflozin” as employed herein refers to tofogliflozin of the above structure as well as pharmaceutically acceptable forms thereof, including hydrates and solvates thereof, and crystalline forms thereof. The compound and methods of its synthesis are described in WO 2007/140191 and WO 2008/013280 for example.

The term “luseogliflozin” as employed herein refers to luseogliflozin of the above structure as well as pharmaceutically acceptable forms thereof, including hydrates and solvates thereof, and crystalline forms thereof.

The term “ertugliflozin” as employed herein refers to ertugliflozin of the above structure as well as pharmaceutically acceptable forms thereof, including hydrates and solvates thereof, and crystalline forms thereof. The compound is described for example in WO 2010/023594.

The term “remogliflozin” as employed herein refers to remogliflozin of the above structure as well as pharmaceutically acceptable forms thereof, including prodrugs of remogliflozin, in particular remogliflozin etabonate, including hydrates and solvates thereof, and crystalline forms thereof. Methods of its synthesis are described in the patent applications EP 1 213 296 and EP 1 354 888 for example.

The term “sergliflozin” as employed herein refers to sergliflozin of the above structure as well as pharmaceutically acceptable forms thereof, including prodrugs of sergliflozin, in particular sergliflozin etabonate, including hydrates and solvates thereof, and crystalline forms thereof. Methods for its manufacture are described in the patent applications EP 1 344 780 and EP 1 489 089 for example.

The compound of formula (16) above, i.e., sotagliflozin, and its manufacture are described for example in WO 2008/042688 or WO 2009/014970.

The term “bexagliflozin” as employed herein refers to bexagliflozin of the above structure as well as pharmaceutically acceptable forms thereof, including hydrates and solvates thereof, and crystalline forms thereof. The compound and methods of its synthesis are described in WO 2009/026537 for example.

The term “TFC-039” as employed herein refers to the above structure as well as pharmaceutically acceptable forms thereof, including hydrates and solvates thereof, and crystalline forms thereof. The compound and methods of its synthesis are described in WO 2012/160218 for example.

The liquid pharmaceutical compositions can be in any suitable form that facilitates easy and effective administration using the delivery system as described herein. For example, the compositions can be in the form of solutions, syrups or suspensions. As described herein with regard to the delivery system, the liquid pharmaceutical compositions can be administered with very precise dosing based upon the bodyweight of the patient. Depending upon the desired characteristics (e.g., viscosity, turbidity level, solubility/suspension level, number and types of active pharmaceutical ingredients, etc.) of the liquid pharmaceutical compositions for a particular form of treatment, a number of other components may also be provided in the compositions in addition to one or more SGLT-2 inhibitor compound(s).

According to a preferred embodiment, the liquid pharmaceutical compositions are in the form of a solution or a suspension. In other words, the pharmaceutical compositions include the one or more SGLT-2 inhibitor compound(s) in solved or suspended form.

With respect to the composition being a suspension, it is preferred when the liquid pharmaceutical composition comprises the one or more SGLT-2 inhibitor compound(s) in the form of solid particles. In this context, the solid particles can have a size, based on the largest dimension of the particles, in the range from 0.01 μm to 150 μm or in the range from 0.1 μm to 15 μm or in the range from 0.2 μm to 10.0 μm or in the range from 0.5 μm to 5 μm.

The SGLT-2 inhibitor(s) can be administered solely as a monotherapy for treatment and/or prevention of a metabolic disorder and/or another medical indication and/or condition to a patient. Alternatively, the form of treatment may include one or more other active agents in addition to the SGLT-2 inhibitor compound(s).

For example, the liquid pharmaceutical compositions can also include one or more SGLT-2 inhibitor(s) combined with any one or more other active pharmaceutical ingredients (APIs), such that both types are administered concomitantly. Alternatively, the liquid pharmaceutical compositions including one or more SGLT-2 inhibitor compound(s) can be administered separately with one or more other APIs. Some non-limiting examples of APIs that can be administered in combination with one or more SGLT-2 inhibitors (either within the same liquid pharmaceutical composition or separately) include the following: diuretics, such as furosemide, torasemide or spironolactone; beta-blockers, such as atenolol or propranolol; calcium-channel blockers, such as diltiazem; ACE inhibitors, such as benazepril, ramipril or enalapril; angiotensin receptors blockers, such as telmisartan; antiarrhythmic agents, such as flecainide; platelet agglutination inhibitors, such as clopidogrel; nonsteroidal anti-inflammatory drugs (NSAIDs), such as aspirin; anticoagulants, such as coumarins (vitamin K antagonists), (low molecular weight) heparin, synthetic pentasaccharide inhibitors of factor Xa, as well as direct factor Xa inhibitors and/or direct thrombin inhibitors; DPP-IV inhibitors; and/or calcium-channel sensitizers and/or positive inotropes, such as pimobendan and/or digitalis alkaloids.

Other solvents, excipients, buffering agents, flavorings and/or other additives, such as preservatives and/or solubilizers, can also be provided in the liquid pharmaceutical compositions as for instance disclosed in WO 2017/032799 and WO 2023/227492/EP 22175413.8. For example, one or more polar organic solvents can be provided in the liquid pharmaceutical compositions, such as ethanol (e.g., in an amount from 0-20 g/100 mL), propylene glycol (propane-1,2-diol) (e.g., in an amount from 0-60 g/100 mL) and/or glycerol (propane-1,2,3-triol) (e.g., in an amount from 0-60 g/mL). In another embodiment, the liquid pharmaceutical composition can be substantially free of organic solvents, i.e. it does not contain any organic solvents at all. Water or an aqueous buffer comprising, e.g., citric acid or a phosphate, can also be provided in the liquid pharmaceutical compositions to achieve any desired pH for the compositions (e.g., in an amount from 0-100 g/100 mL, preferably 30-100 g/100 mL). For example, the liquid pharmaceutical compositions can be prepared to have pH values ranging from 3 to 9.

It was surprisingly found that the solvents take influence on the dosability and applicability of the liquid pharmaceutical compositions and that certain solvents or combinations of solvents make administration of very small volumes easier and more precise.

Particularly good results are achieved when the liquid pharmaceutical compositions include at least two or three organic polar solvents, with propylene glycol (propane-1,2-diol) being one of at least two or three organic polar solvents. Such compositions are disclosed in WO 2017/032799. Compositions 1 to 6 as disclosed in WO 2017/032799 are hereby incorporated by reference.

Preferably, the liquid pharmaceutical compositions include propylene glycol in an amount in the range from 10 to 60 g/100 mL, especially in the range from 35 to 60 g/100 mL, preferably in the range from 50 to 60 g/100 mL, and at least one or two more organic polar solvents being selected from ethanol and/or glycerol (propane-1,2,3-triol). With respect to the further polar organic solvents it is further preferred, when the liquid pharmaceutical compositions include ethanol in an amount in the range from 1 to 20 g/100 mL, especially in the range from 1 to 15 g/100 mL, preferably in the range from 1 to 10 g/100 mL, more preferably in the range from 1 to 8 g/100 mL, and/or glycerol in an amount in the range from 1 to 60 g/100 mL, especially in the range from 1 to 52 g/100 mL.

According to a particularly preferred embodiment, the liquid pharmaceutical compositions include propylene glycol in an amount in the range from 10 to 60 g/100 mL, especially in the range from 35 to 60 g/100 mL, preferably in the range from 50 to 60 g/100 mL, and ethanol in an amount in the range from 1 to 20 g/100 mL, especially in the range from 2 to 15 g/100 mL, preferably in the range from 3 to 10 g/100 mL, particularly preferably in the range from 5 to 8 g/100 mL. Composition 5 according to WO 2017/032799, said composition comprising propylene glycol in an amount of 60 g/100 mL and ethanol in an amount of 8 g/100 mL, shows particularly precise dosability in delivery systems as described in paragraphs Error! Reference source not found. to Error! Reference source not found. of this specification.

In yet another aspect, the liquid pharmaceutical compositions can include ethanol as the only organic polar solvent. According to this embodiment, the liquid pharmaceutical compositions contain no more than 20 g/100 mL of ethanol, preferably no more than 15 g/100 mL of ethanol, more preferably no more than 10 g/100 mL of ethanol. Preferably, ethanol is present in the liquid pharmaceutical compositions in an amount in the range from 1 to 20 g/100 mL, preferably in the range from 2 to 15 g/100 mL, more preferably in the range from 5 to 10 g/100 mL. According to a particularly preferred embodiment, ethanol is present in the liquid pharmaceutical compositions in an amount of 8 g/100 mL. Examples for such compositions are disclosed in EP 22175413.8 and WO 2023/227492 as Compositions 1 to 4. Compositions 1 to 4 as disclosed in EP 22175413.8 and WO 2023/227492 are hereby incorporated by reference.

In yet another aspect, the liquid pharmaceutical compositions do not contain any organic polar solvent, especially organic solvents as described in paragraphs Error! Reference source not found. to Error! Reference source not found. In case no organic polar solvent is present, the solvent of the liquid pharmaceutical composition is 100% water, preferably in the form of aqueous buffer. Examples for suitable compositions being free of organic polar solvents are described in EP 22175413.8 and WO 2023/227492 as Compositions 5 to 12. Compositions 5 to 12 as disclosed in EP 22175413.8 and WO 2023/227492 are hereby incorporated by reference.

The liquid pharmaceutical compositions can also include one or more solubilizing agents including, without limitation, surfactants, anionic surfactants, non-ionic surfactants, hydrogenated castor oils, polyoxyethylene-polyoxypropylene block copolymers, polyethylene glycols, propylene glycol derivatives. Some specific non-limiting examples of suitable solubilizing agents include sodium dodecyl sulphate (SDS), Cremophor RH 40 (PEG-40 Hydrogenated Castor Oil, Macrogol glycerol hydroxystearate 40), polysorbate 20, Lutrol F 68 (Poloxamer 188), PEG 200, PEG 300, PEG 400, propylene glycol monolaurate, and Kollidon 12 (povidone) (e.g., in an amount from 0-50 g/100 mL, preferably 1-50 g/100 mL).

According to a preferred embodiment of this aspect, the liquid pharmaceutical compositions contain one or more solubilizing agent, particularly wherein the solubilizing agents are selected from sodium dodecyl sulphate (SDS), Cremophor RH 40 (PEG-40 Hydrogenated Castor Oil, Macrogol glycerol hydroxystearate 40), polysorbate 20, Lutrol F 68 (Poloxamer 188), PEG 200, PEG 300, PEG 400, propylene glycol monolaurate, Kollidon 12 (povidone), and combinations thereof.

Preferably, the total amount of solubilizing agents in the liquid pharmaceutical compositions is from 1 to 50 g/100 mL, especially from 1 to 45 g/100 mL, preferably from 1 to 40 g/100 mL, more preferably from 1 to 35 g/100 mL, even more preferably from 1 to 30 g/mL, even more preferably from 1 to 25 g/100 mL, most preferably from 5 to 25 g/100 mL.

In yet another aspect, the liquid pharmaceutical compositions as described herein contain two or more, preferably two solubilizing agents. In this context it is preferred when the solubilizing agents are Kollidon 12 (povidone) as well as PEG 200, PEG 300, or PEG 400, more preferably Kollidon 12 (povidone) and PEG 300.

According to a further embodiment, particularly if the composition is a suspension, the liquid pharmaceutical compositions contain a liquid vehicle for carrying solid particles comprising the one or more SGLT-2 inhibitor compound(s). The liquid vehicle can be aqueous or organic, preferably aqueous. According to a particularly preferred embodiment, the liquid vehicle is water or aqueous buffer.

In yet another aspect, one or more suspending agents can be provided in the liquid pharmaceutical compositions. Examples for suitable suspending agents are viscosity-enhancing agents, e.g. cellulose derivatives or gel forming agents, and/or thixotropic agents, e.g. gel forming polysaccharides or natural gums.

One or more viscosity-enhancing agents can also be provided in the liquid pharmaceutical compositions including, without limitation, inorganic gel forming agents, organic gel forming agents, cellulose derivatives. Some specific non-limiting examples of suitable viscosity-enhancing agents include hydroxyl ethyl cellulose, hydroxyl propyl methyl cellulose, methyl cellulose, carboxyl methyl cellulose and silicon dioxide.

One or more thixotropic agents can be provided in the liquid pharmaceutical compositions, including without limitation, synthetic or natural gums and mucilage. Some specific non-limiting examples of suitable thixotropic agents include tragacanth, xanthan, or alginate.

One or more flavoring agents (including flavors and/or sweeteners) can be provided in the liquid pharmaceutical compositions (particularly for compositions used for oral administration). Non-limiting examples of flavoring agents that can be provided in the compositions include honey flavor, lime/salvia flavor, jasmine flavor, lavender flavor, peppermint flavor, raspberry flavor, lemon flavor, herbs flavor, meat flavor, artificial beef flavor, saccharine, stevia, and aspartame.

The concentration of API(s) including SGLT-2 inhibitor(s) in the liquid pharmaceutical compositions can vary depending upon a number of factors including, without limitation, required daily dosing amount for a particular type of patient, solubility characteristics of the API(s) within solution combined with other components in solution, etc. The concentration of one or more SGLT-2 inhibitor compound(s) in the liquid pharmaceutical compositions can range from 0.1 mg/mL to 20 mg/mL or greater, e.g., from 1.2 mg/mL to 15 mg/mL, or from 1 mg/mL to 5 mg/mL, or from 5 mg/mL to 20 mg/mL, or from 10 mg/mL to 20 mg/mL, or from 12 mg/mL to 18 mg/mL. In an example embodiment (e.g., for treatments of felines), the concentration of SGLT-2 inhibitor compound(s) within the liquid pharmaceutical compositions can be 15 mg/mL. In a further example embodiment (e.g., for treatments of canines), the concentration of SGLT-2 inhibitor compound(s) within the liquid pharmaceutical compositions can be 1.2 mg/mL. In another further example embodiment (e.g., for treatments of equines), the concentration of SGLT-2 inhibitor compound(s) within the liquid pharmaceutical compositions can be 15 mg/mL.

### Dosage and Administration

A dosage amount of one or more SGLT-2 inhibitor(s) to be administered to a patient can be determined based upon a number of factors, including patient/mammal type, size/bodyweight of patient, specific type(s) of metabolic disorder and/or other medical condition(s) to be prevented and/or treated, etc.

The liquid pharmaceutical compositions and delivery systems for administering such compositions as described herein are suitable for use in treating and/or preventing one or more metabolic disorders and/or other medical indications in a subject in any suitable patient. The patient is preferably an animal, more preferably a non-human mammal, such as an equine (e.g., a horse), a canine (e.g., a dog), a feline (e.g., a cat) or a ruminant (e.g., a cow). The embodiments described herein are particularly useful for treatments related to smaller companion animals, such as felines and (small) canines.

Generally, a dosage amount of one or more SGLT-2 inhibitor(s) to be administered to a patient will be in the range from 0.01 to 10 mg/kg BW (patient bodyweight) per day. The dose can be administered once per day or a plurality of times per day, such as twice per day (e.g., once per every 12 hours), three times per day, etc. Depending upon the bodyweight of the patient, and concentration of the one or more SGLT-2 inhibitor compound(s) in the liquid pharmaceutical compositions, the dosage amount can be very small (e.g., in the order of 1 mL or less). Thus, it is important to have an effective delivery system that can provide a precise dosage amount.

For example, the one or more SGLT-2 inhibitor compound(s) can be administered in daily doses from 0.01-10 mg/kg BW, or from 0.01-5 mg/kg BW, or from 0.01-4 mg/kg BW, or from 0.01-3 mg/kg BW, or from 0.01-2 mg/kg BW, or from 0.01-1.5 mg/kg BW, or from 0.01-1 mg/kg BW, or from 0.01-0.75 mg/kg BW, or from 0.01-0.5 mg/kg BW, or from 0.01-0.4 mg/kg BW, or from 0.01-0.3 mg/kg BW; or from 0.1 to 5.0 mg/kg BW, or from 0.1 to 3.0 mg/kg BW, or from 0.2 to 2.0 mg/kg BW, or from 0.1 to 1 mg/kg BW, or from 0.02-0.5 mg/kg BW, or from 0.03-0.4 mg/kg BW, or from 0.03-0.3 mg/kg BW.

An SGLT-2 inhibitor can be administered, such that an appropriate blood plasma concentration of the SGLT-2 inhibitor is achieved (e.g. a maximal blood plasma concentration, or blood plasma concentration after a given time, e.g. 4, 8, 12 or 24 hours after oral administration, preferably about 8 hours after oral administration). E.g., for Velagliflozin, the blood plasma concentration (e.g., maximal blood plasma concentration or blood plasma concentration after said given time after oral administration) may be within the range 2 to 4000 nM, e.g., 20 to 3000, or e.g., 40 to 2000 nM. Preferably, following administration and the time required for an SGLT-2 inhibitor to reach the bloodstream, such levels are maintained in the blood over a time interval of at least 12 hours, more preferably at least 18 hours, most preferably at least 24 hours.

Preferably, according to embodiments described herein, the liquid pharmaceutical compositions are administered orally. Using the delivery system described herein, the liquid pharmaceutical compositions including SGLT-2 inhibitor compound(s) can be administered directly to the patient's mouth or together with the animal's food or drink (e.g., with its drinking water or the like). Alternatively, the liquid pharmaceutical compositions can also be administered parenterally, or by any other suitable route of administration (e.g., rectally) via the delivery system described herein.

### Metabolic Disorders/Other Medical Conditions

The liquid pharmaceutical compositions that include one or more SGLT-2 inhibitor(s) as described herein can be used, in combination with the delivery system as described herein, for treatment and/or prevention of various metabolic disorders and/or other medical indications for various types of patients.

For example, the liquid pharmaceutical compositions including one or more SGLT-2 inhibitor compound(s) as described herein can be used for treatment and/or prevention of one or more metabolic disorders (or conditions associated with such metabolic disorders) in equine patients (e.g., horses) including, without limitation, insulin resistance, hyperinsulinemia, impaired glucose tolerance, dyslipidemia, dysadipokinemia, subclinical inflammation, systemic inflammation, low grade systemic inflammation, which also comprises adipose tissue, obesity, regional adiposity, laminitis, vascular dysfunction, hypertension, hepatic lipidosis, atherosclerosis, hyperadrenocorticism, Pituitary Pars Intermedia Dysfunction and/or Equine Metabolic Syndrome.

The liquid pharmaceutical compositions including one or more SGLT-2 inhibitor compound(s) as described herein can also be used for treatment and/or prevention of one or more metabolic disorders (or conditions associated with such metabolic disorders) in canine patients (e.g., dogs) including, without limitation, diabetes, pre-diabetes, obesity and/or any disorder, disease, condition or symptom associated with one or more of those disorders. In particular, the metabolic disorder may be hyperglycaemia, impaired glucose tolerance, insulin resistance, insulin dependent diabetes and/or hepatic lipidosis. Further relevant metabolic disorders include hyperinsulinaemia, impaired glucose tolerance, ketosis (in particular ketoacidosis), hyperlipidaemia, dyslipidemia, elevated blood levels of fatty acids and/or of glycerol, Syndrome X (metabolic syndrome), and/or inflammation of the pancreas, low grade systemic inflammation, inflammation of adipose tissue. The liquid pharmaceutical compositions including one or more SGLT-2 inhibitor compounds as described herein can be further used for treatment and/or prevention of one or more other medical conditions in canine patients (e.g., dogs) including, without limitation, one or more cardiac diseases. In particular, the one or more cardiac diseases can include, without limitation, heart failure; congestive heart failure; asymptomatic/preclinical/occult heart failure; heart failure due to (myxomatous) mitral valve disease [(M)MVD]; congestive heart failure due to (myxomatous) mitral valve disease [(M)MVD]; asymptomatic/preclinical/occult heart failure due to (myxomatous) mitral valve disease [(M)MVD]; (myxomatous) mitral valve disease [(M)MVD]; clinically overt (myxomatous) mitral valve disease [(M)MVD]; asymptomatic/preclinical/occult (myxomatous) mitral valve disease [(M)MVD]; heart failure due to dilated cardiomyopathy (DCM); congestive heart failure due to dilated cardiomyopathy (DCM); asymptomatic/preclinical/occult heart failure due to dilated cardiomyopathy (DCM); dilated cardiomyopathy (DCM); clinically overt dilated cardiomyopathy (DCM); asymptomatic/preclinical/occult dilated cardiomyopathy (DCM); aortic stenosis (valvular, supravalvular and/or subvalvular).

Further, the liquid pharmaceutical compositions including one or more SGLT-2 inhibitor compound(s) as described herein can be used for treatment and/or prevention of one or more metabolic disorders (or conditions associated with such metabolic disorders) in feline patients (e.g., cats) including, without limitation, diabetes, pre-diabetes, diabetes mellitus type 2, acromegaly, diabetes with elevated IGF-1 concentration, obesity and/or any disorder, disease, condition or symptom associated with one or more of those disorders, hyperglycemia, insulin resistance, diabetes and/or hepatic lipidosis, hyperinsulinemia, impaired glucose tolerance, ketosis (in particular ketoacidosis), hyperlipidemia, elevated blood levels of fatty acids and/or of glycerol, Syndrome X (metabolic syndrome), atherosclerosis, inflammation of the pancreas, inflammation of adipose tissue and/or loss of pancreatic beta cell function. The liquid pharmaceutical compositions including one or more SGLT-2 inhibitor compound(s) as described herein can be used for treatment and/or prevention of one or more other medical conditions in feline patients (e.g., cats) including, without limitation, one or more cardiac diseases. In particular, the one or more cardiac diseases can include, without limitation, heart failure, heart failure due to one or more cardiomyopathies, heart failure due to hypertrophic cardiomyopathy (HCM), heart failure due to restrictive cardiomyopathy (RCM), heart failure due to dilated cardiomyopathy (DCM), heart failure due to unclassified cardiomyopathy (UCM), heart failure due to arrythmogenic right ventricular cardiomyopathy (ARVC), hypertrophic cardiomyopathy (HCM), restrictive cardiomyopathy (RCM), dilated cardiomyopathy (DCM), unclassified cardiomyopathy (UCM), and/or arrythmogenic right ventricular cardiomyopathy (ARVC); preferably selected from the group consisting of: heart failure due to one or more cardiomyopathies, heart failure due to hypertrophic cardiomyopathy (HCM), hypertrophic cardiomyopathy (HCM).

Further, the liquid pharmaceutical compositions including one or more SGLT-2 inhibitor compound(s) as described herein can be used for treatment and/or prevention of one or more renal diseases in non-human mammals, such as feline and/or canine patients (e.g., cats and/or dogs) including, without limitation, renal dysplasia, glomerulopathy, polycystic kidney disease, amyloidosis, tubulo-nephritis/tubulointerstitial nephritis (TIN), acute kidney disease, chronic kidney disease.

Further, the liquid pharmaceutical compositions including one or more SGLT-2 inhibitor compound(s) as described herein can be used for treatment and/or prevention of hypertension in non-human mammals, such as feline and/or canine patients (e.g., cats and/or dogs) including, without limitation, situational hypertension, secondary hypertension and idiopathic hypertension, wherein the secondary hypertension can be selected from the group consisting of hypertension associated with chronic kidney disease (CKD), diabetes, obesity, heart disease, endocrine disease, such as Cushing's disease, hyperthyroidism, acromegaly, and elevated blood pressure (BP) induced by medicaments, preferably by glucocorticoids, mineralocorticoids, erythropoiesis-stimulating agents, ephedrine and/or high dose sodium chloride.

Further, the liquid pharmaceutical compositions including one or more SGLT-2 inhibitor compound(s) as described herein can be used for drying-off of non-human mammals, such as ruminants, including, without limitation, improving and/or facilitating the drying-off, reducing the milk production, decreasing milk accumulation and/or engorgement in the udder and/or mammary gland, decreasing the discomfort associated with udder engorgement, such as increasing the daily lying time and/or reduction of stress, decreasing milk leakage after drying-off, and/or decreasing the incidence of intra-mammary infections (IMI), such as mastitis and/or metritis.

### Delivery System

The delivery system that facilitates administration of a liquid pharmaceutical composition as described herein to a patient includes a storage container for receiving and storing the composition and a syringe that effectively draws a precise dosage volume of the composition from the container and delivers the dosage volume to the patient (directly or indirectly).

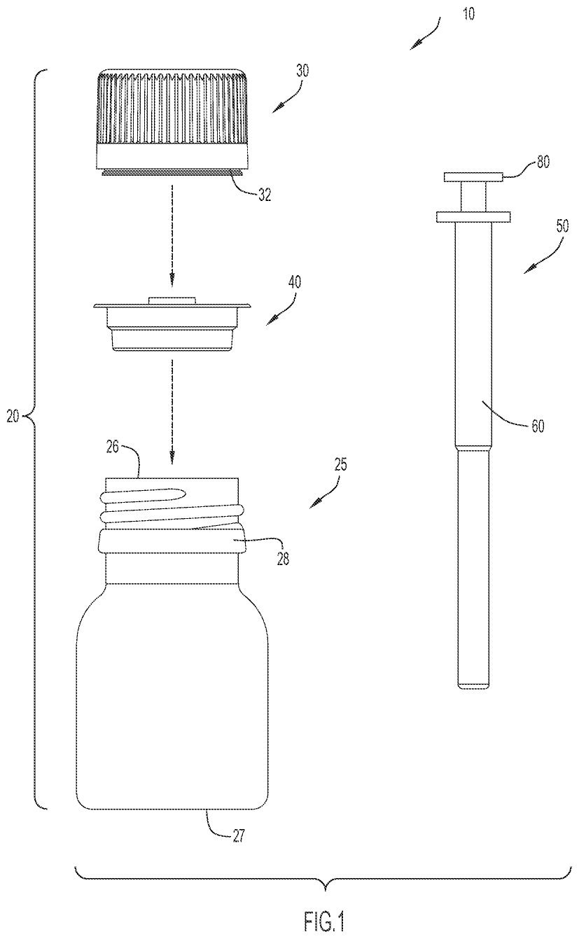

Referring to FIGS. 1-7, a delivery system 10 for the liquid pharmaceutical composition includes a container 20 and a syringe 50. The container 20 includes a storage vessel 25 with a hollow interior, an open upper end 26, and an opposing lower closed end 27, where the storage vessel 25 has a suitable geometry and sufficient interior volume to store a suitable number of doses of liquid pharmaceutical composition prior to use. For example, the storage vessel can have a volume ranging from 10 mL to 60 mL, preferably from 15 mL to 45 mL, so as to facilitate a sufficient daily dosage amount of liquid pharmaceutical composition for at least 14 days, preferably at least 28 days or 30 days, where the SGLT-2 concentration within the liquid pharmaceutical composition can range from 1.2 mg/mL to 15 mg/mL (e.g., 1.2 mg/mL for canines, 15 mg/mL for felines, 15 mg/mL for equines).

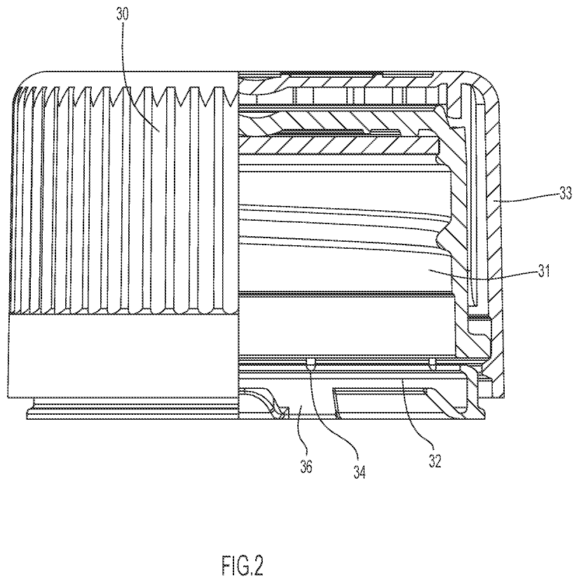

The container 20 further includes a lid or cap 30 that is removably secured to the open upper end 26 of the vessel 25 (e.g., via a threaded engagement between cap and vessel open end, as shown in the partial cross-sectional view of the cap as shown in FIG. 2). Each of the storage vessel 25 and cap 30 are constructed of one or more polymers and/or other suitable materials that are relatively inert and non-reactive to any of the components of the liquid pharmaceutical composition stored within the container 20. For example, each of the storage vessel and cap can be constructed of one or more polyolefin materials. In an example embodiment, the storage vessel is constructed of a polymer material comprising, preferably consisting of, polyethylene (e.g., high density polyethylene, or HDPE), while the cap is constructed of a polymer material comprising, preferably consisting of, polypropylene and polyethylene, particularly preferably polypropylene. As is known, high density polyethylene (HDPE) has a linear polymer chain structure and is opaque, whereas low density polyethylene (LDPE) has a branched polymer chain structure and is transparent or translucent. While HDPE and LDPE have similar properties, HDPE has a greater hardness or rigidness and is more tear resistant/abrasion resistant in relation to LDPE, and LDPE is a softer, more flexible material in relation to HDPE.

The cap 30 includes an outer portion 33 comprising, preferably consisting of, polypropylene, and an inner portion 31 comprising, preferably consisting of, polyethylene (e.g., high density polyethylene, or HDPE). The cap inner portion 31 includes an inner threading that engages in a threaded connection with a corresponding outer threading at the open upper end 26 of the vessel 25. The inner portion 31 can be secured or fused in the outer portion 33 in any suitable manner (e.g., via adhesion, welding a co-molding process, etc.). A liner (e.g., comprising, preferably consisting of, polyethylene) can be provided at an upper interior surface of the inner portion 31 so as to engage with the vessel 25 at its open upper end 26 and provide an effective fluid tight seal at such interface. The cap can further include a tamper-evident element that provides an indication of whether the container has been opened (i.e., cap partially or fully removed from the storage vessel) prior to initial use of the delivery system). In particular, the inner portion 31 of the cap 30 includes a detachable ring 32 that is removably connected at an open end of the cap 30 directly below the inner threading of the cap (where the inner threading of the cap engages with the corresponding outer threading at the open upper end 26 of the vessel 25). The detachable ring 32 can be connected, e.g., via break-away tabs 34, with the inner portion 31 of the cap 30 at the lower end of the cap.

During initial installation or connection of the cap 30 with the open upper end 26 of the vessel 25, the cap 30 is connected with the vessel upper end via the corresponding threading, where the cap is rotated (e.g., in a clockwise direction) and moved downward onto the vessel upper end to close the container 20. The detachable ring 32 also moves downward along the vessel upper end and moves or rides over a ledge 28 at the vessel open upper end 26. The detachable ring 32 further includes one or more inwardly extending and upwardly angled (i.e., angled toward the closed end of the cap) stop members 36. The ring 32 and one or more stop members 36 are configured to allow the ring 32 and stop member(s) 36 to ride over and pass the ledge 28 when rotated downward (e.g., in a clockwise direction) onto the vessel 25. However, when the ring 32 is situated beneath the ledge 28 (i.e., between the ledge and the lower closed end 27 of the vessel 25), the stop member(s) 36 prevent upward movement of the ring 32 beyond the ledge 28. The cap 30 can still be rotated upward (rotated, e.g., in a counter-clockwise direction) and removed from the open upper end 26 of the vessel 25 due to the break-away tabs 34 disengaging the ring 32 from the inner portion 31 of the cap 30 after the cap is rotated a selected distance to being removed from the vessel 25 (e.g., partially disengaged or fully disengaged/removed from the vessel). Separation of the ring 32 from the cap 30 provides an indication that the container 20 has been at least partially opened and thus used (at least once) or tampered with in some manner (potentially compromising the liquid pharmaceutical composition within the container) prior to initial use.

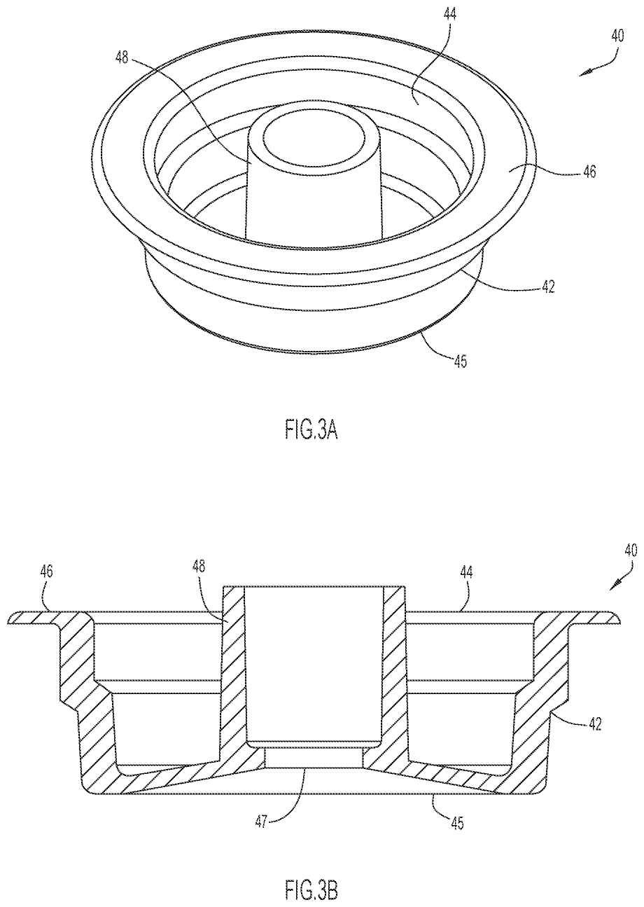

The container 20 can further include an adaptor 40 that is secured at the top or upper open end of the vessel 25. The adaptor can be constructed of a polymer or other suitable material, such as a polyolefin material. In an example embodiment, the adaptor 40 is constructed of a polymer material comprising, preferably consisting of, polyethylene (e.g., low density polyethylene, or LDPE). Referring to FIGS. 3A and 3B, the adaptor 40 comprises a hollow and elongated (e.g., generally cylindrical) main member 42 that is suitably dimensioned to substantially fit within the open upper end 26 of the vessel 25. At least a portion of the main member 42 of the adaptor 40 has an outer diameter that is substantially similar and can be slightly greater in relation to the inner diameter (I.D.) at the open upper end 26 of the vessel 25. The dimensions of the adaptor and open upper end of the container vessel, combined with materials of construction for both components (e.g., adaptor formed of LDPE, container vessel formed of HDPE), ensures a frictionally and fluid tight fit between the two components when the adaptor 40 is installed in the vessel open upper end 26 with the outer wall portions of the main member 42 engaging the inner wall portions of the vessel 25 at its open upper end. The main member 42 includes an open upper end 44 and an end wall at the opposing lower end 45. A circular flange 46 is disposed at the open upper end 44 of the main member 42, where the flange 46 is greater in transverse cross-sectional dimension in relation to the main member 42. The flange 46 extends outward at the main member upper end 44 and has a diameter that generally corresponds with an outer diameter of the vessel 25 at its open upper end 26. Thus, when the adaptor 40 is connected with the vessel 25, the flange 46 is secured at and rests upon the open upper end 26 of the vessel 25. The adaptor 40 is further connected with the vessel 25 in a suitable manner to provide a fluid-tight seal between the two components at the vessel open end.

The end wall at the opposing lower end 45 of the main member 42 includes a central opening 47 that is smaller in size in relation to the opening at the upper end 44. Extending from the end wall at the lower end 45 in a lengthwise direction within the hollow main member 42 is an elongated and hollow (e.g., cylindrical) inner connection member 48, where the inner connection member 48 communicates (i.e., is in alignment) in a fluid tight manner with the central opening at the lower end wall of the lower end 45 and further extends within the outer, hollow main member 42 to the open upper end 44. The inner connection member 48 is open at its upper end, and the inner connection member upper end is generally coplanar or flush with or, alternatively, extends slightly beyond, the flange 46. Thus, when the adaptor 40 is secured with the vessel 25 at the vessel open upper end 26, the adaptor provides an outlet through the elongated inner connection member 48 for the liquid pharmaceutical composition to be withdrawn from the vessel interior. As described herein in relation to the syringe 50, the transverse cross-section (e.g., diameter) of the hollow inner connection member 48 is suitably dimensioned to engage in a releasable, frictional and fluid-tight manner with a corresponding end of the syringe.

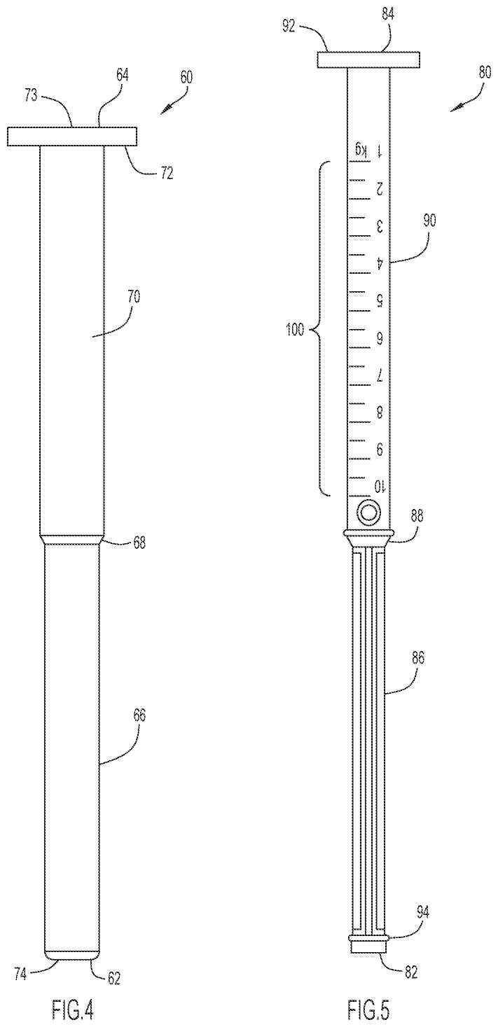

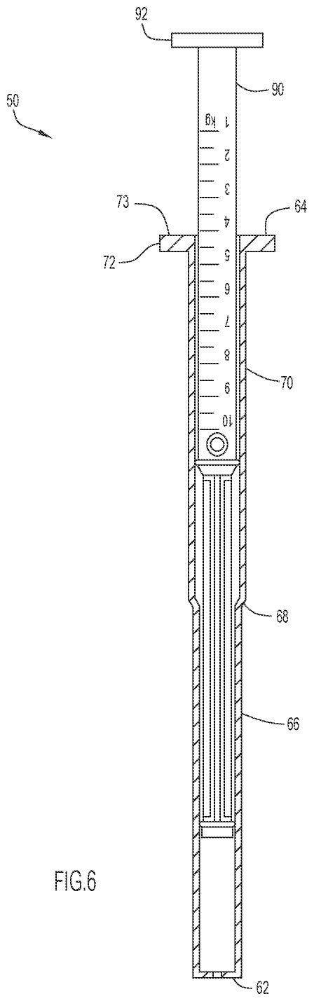

Referring to FIGS. 4 and 5, the syringe 50 of the delivery system 10 includes a barrel 60 and a plunger 80, where the plunger 80 is suitably dimensioned such that a portion of the plunger is slidably received and movable in a telescoping manner within the barrel to facilitate drawing of fluid from the container into the barrel and forcing fluid flow from the barrel via the plunger in use. Each of the syringe barrel and plunger can be constructed of any suitable polymers and/or other materials that are relatively inert and non-reactive to any components of the liquid pharmaceutical compositions with which the syringe interacts during use. For example, each of the barrel and plunger of the syringe can be made of the same or different polyolefin materials. In a preferred embodiment, the syringe barrel is constructed of polymer material comprising, preferably consisting of, polyethylene (e.g., low density polyethylene or LDPE), while the syringe plunger is constructed of a polymer material comprising, preferably consisting of, polystyrene. The choice of materials for plunger and barrel has surprising effects on the dosing properties of the syringe. A syringe barrel constructed of LDPE and a plunger constructed of polystyrene in combination lead to an improved, particularly a more precise application or delivery of very small volumes of the liquid pharmaceutical composition.

The syringe barrel 60 is hollow and has an elongated and generally cylindrical configuration including a lower or forward end 62 through which the solution is drawn (via the plunger 80) from the vessel 25 of the container 20. The barrel 60 further has an open upper or rear end 64 that is suitably dimensioned to receive a portion of the plunger 80. The cross-sectional dimension or diameter of the barrel 60 changes along its length, where the barrel includes a first portion 66 extending from the forward end 62 to a transition section 68 in which the barrel increases in cross-sectional dimension or diameter at the transition section. A second portion 70 of the barrel 60 extends from the transition section 68 to the open rear end 64. The second portion 70 has a cross-sectional dimension or diameter (i.e., a second cross-sectional dimension) that is greater than the cross-sectional dimension or diameter (i.e., first cross-sectional dimension) of the first portion 66. A finger gripping flange 72 is provided at the open rear end 64 and has a cross-sectional dimension that is greater than the cross-sectional dimension of the open rear end 64.

The barrel forward end 62 includes an end wall 74 with a small central opening through the end wall 74. The opening provided through the end wall 74 at the barrel forward end 62 is smaller in size in relation to the opening at the barrel rear end 64. The barrel first portion 66 also has a suitable outer wall diameter (O.D.) that is substantially similar and can be slightly greater in relation to an inner diameter (I.D.) of the elongated inner connection member 48 of the adaptor 40 at the adaptor upper end. This facilitates a frictional and fluid tight connection between the barrel first portion 66 and the adaptor inner connection member 48 when the barrel forward end 62 is inserted into the inner connection member 48. This facilitates effective transfer of the liquid pharmaceutical composition from the container 20 into the hollow interior of the barrel 60 while preventing fluid leakage between these two components. In certain embodiments, the barrel first portion at the barrel forward end can have a unique outer cross-sectional geometry (e.g., tapered, multi-faceted, etc.) that matches an internal cross-sectional geometry of the elongated inner connection member for the adaptor.

The close dimensional correspondence between O.D. of the barrel first portion at the barrel forward end and I.D. of the adaptor inner connection member as well as, optionally, any corresponding or matching geometries at the engaging surfaces between barrel forward end and adaptor inner connection member provides a key-like connection that requires use of the syringe 50 with the container 20 in order to ensure a secure and fluid tight connection at their interfaces and effective transfer of liquid pharmaceutical composition into the syringe barrel. In other words, the key-like connection between the syringe barrel and the container, via the adaptor, ensures only the specific syringe can be used effectively with the container containing the particular liquid pharmaceutical composition, and another syringe cannot be exchanged for the syringe configured for use with the medicament provided in the corresponding container. Such key-like features can be unique for the specific syringe and its paired container with specified liquid pharmaceutical composition. This limits or prevents using a specific syringe of the delivery system with another container that might include a different liquid pharmaceutical composition that is not configured for use with the specific syringe.

The plunger 80 comprises an elongated member that is suitably dimensioned to be substantially received within the barrel 60 via the open rear end 64 and facilitate a slidable, telescoping movement of the plunger within the barrel during use. The plunger 80 includes an upper or rear end 84 and a lower or forward end 82, where the plunger forward end 82 is provided within the barrel and can extend with the hollow interior of the barrel within the barrel first portion 66 and the barrel second portion 70. The plunger includes a first portion 86 that extends from the plunger forward end 82 to a transition section 88, and a second portion 90 that extends from the transition section 88 to the plunger rear end 84. Similar to the barrel 60, the plunger second portion 90 has a transverse cross-section (e.g., diameter) that is greater than a transverse cross-section of the plunger first portion 86. A finger gripping flange 92 is provided at the plunger rear end 84, where the flange 92 extends transversely beyond the cross-sectional dimension of the plunger rear end 84 so as to provide a gripping surface during use of the syringe 50 (e.g., moving the plunger within the barrel).

The plunger 80 has a longitudinal or lengthwise dimension that is at least substantially the same as, and preferably greater than, the longitudinal or lengthwise dimension of the barrel 60. This facilitates full receipt of the plunger into the barrel, where the plunger forward end can engage with the forward end wall of the barrel. In addition, when the plunger has a greater length than the barrel, a small portion of the plunger at its rear end extends beyond the rear end of the barrel when the plunger is fully received within the barrel (i.e., plunger forward end in contact with barrel forward end). In such an embodiment, the plunger flange always extends a suitable distance beyond barrel flange (thus permitting case of gripping of the plunger flange for drawing a portion of the plunger from the barrel).

Each of the plunger and barrel are suitably dimensioned to facilitate withdrawal of a sufficient amount of liquid within the barrel to ensure an adequate dose of liquid pharmaceutical composition to the patient. The forward end 82 of the plunger 80 includes a stopper member 94 that is dimensioned so as to provide a frictionally tight and fluid tight engagement along the inner walls of the barrel first portion 66. The stopper member 94 can be formed of the same material as the rest of the plunger 80 (e.g., polystyrene), including first portion 86, transition section 88, and second portion 90. The stopper member 94 has a transverse cross-section that is greater than the internal transverse cross-section (e.g., internal diameter) of the barrel transition section 68. During installation of the plunger within the barrel, the plunger 80 can be inserted within the barrel 60 with the plunger forward end 82 with stopper member 94 being forced downward past the barrel transition section 68 and toward the barrel forward end 62. During withdrawal of the plunger from the barrel, the barrel transition section 68 provides a minor abutment for the plunger forward end 82 with stopper member 94 so as to provide an indication to the user of a stopping point at which the plunger should not be withdrawn further from the barrel. Thus, the maximum fluid volume within the barrel 60 is limited to the volume defined by the barrel first portion 66.

The syringe 50 is configured so as to provide certain beneficial features for withdrawing very small, discrete and precise dosages of the liquid pharmaceutical composition from the container 20 depending upon the dosage amount required for a patient. For example, when a patient has a relatively low bodyweight (e.g., a feline or a small canine), the volume of liquid pharmaceutical composition that must be administered may need to be very small. The volume of the liquid pharmaceutical composition will depend upon dosage required for treatment (based upon mg of active ingredient/kg BW of patient), the concentration of active ingredient (e.g., SGLT-2 inhibitor) in the liquid pharmaceutical composition, and the bodyweight of the patient. Consider a feline to be treated for a metabolic disorder, where the feline weighs about 2 kg, a single dosage is required to be 1.0 mg/kg BW, and the concentration of the liquid pharmaceutical composition is 15 mg/mL. The single dosage to the feline (e.g., administered orally) for this scenario would be about 0.133 mL. Providing such a small volumetric dosage amount within the barrel of a conventional syringe would be very difficult.

The syringe 50 can be configured such that the barrel volume defined at the first portion 66 is limited to no greater than 3 mL, preferably no greater than 2 mL, more preferably no greater than 1 mL, e.g., no greater than 0.9 mL, or no greater than 0.8 mL, or no greater than 0.7 mL, or no greater than 0.6 mL, or no greater than 0.5 mL, or preferably no greater than 0.4 mL. Particularly, the syringe 50 can be configured such that the barrel volume defined at the first portion 66 is in the range from 0.01 mL to 3 mL, preferably in the range from 0.01 mL to 2 mL, more preferably in the range from 0.01 mL to 1 mL, especially in the range from 0.02 mL to 0.9 mL, further preferred in the range from 0.03 mL to 0.8 mL, more preferred in the range from 0.04 mL to 0.7 mL, most preferred in the range from 0.05 mL to 0.6 mL. The dosage increments for the syringe, which are based upon bodyweight of the patient, could be on a graduated scale of as many as 10 or more increments. Thus, dosage volumes could be as small as 0.05 mL or even lower. According to a preferred embodiment, the dosage volumes will be in the range from 0.01 mL to 3 mL, preferably in the range from 0.01 mL to 2 mL, more preferably in the range from 0.01 mL to 1 mL, especially in the range from 0.02 mL to 0.9 mL, further preferred in the range from 0.03 mL to 0.8 mL, more preferred in the range from 0.04 mL to 0.7 mL, most preferred in the range from 0.05 mL to 0.6 mL. In example embodiments, the dosage volumes for the syringe will be in the range from 0.05 mL to 0.6 mL. The configuration of the syringe 50 is such that each dosage increment can be easily selected depending upon the particular scenario of treatment for a patient.

Nevertheless, the barrel volume defined at the first portion 66 can be selected depending on the body weight, size and dosage amount required for the patient. Therefore, in yet another aspect, the syringe 50 can be configured such that the barrel volume defined at the first portion 66 is in the range from 5 mL to 40 mL, preferably in the range from 10 mL to 30 mL, more preferably in the range from 15 mL to 25 mL. According to this embodiment, the dosage volumes will be in the range from 5 mL to 40 mL, preferably in the range from 10 mL to 30 mL, more preferably in the range from 15 mL to 25 mL. Such dosage volumes and configurations of the syringe 50 are particularly suitable for the treatment of equines.

In particular, the barrel first portion 66, which defines the maximum volume that can be withdrawn from the container 20 into the syringe 50, has a length that is much greater than its internal diameter (I.D.) or internal transverse cross-section. By increasing the length of the internal volume in relation to the internal diameter of the barrel portion that receives fluid, a drawing of fluid within the barrel by the plunger in volumetric increments that are very small with high precision and granularity is made possible, since the plunger can be withdrawn a greater length from the barrel while minimizing the amount of fluid that is drawn into the barrel internal volume with each incremental displacement of the plunger from the barrel. Preferably, the barrel first portion 66 has a length to internal diameter (or internal transverse cross-section) ratio, referred to as L/D, of at least 5. For example, the barrel first portion 66 can have an L/D ratio from 5 to 20, or from 7 to 15, or from 9 to 14. In a specific example embodiment, the L/D ratio for the barrel first portion is about 12.2 (L/D=12.2). This can also provide the effect of increasing the distance between demarcations that define volumetric increments on a graduation scale provided as indicia on the syringe (i.e., making it easier for a user to visually interpret a correct amount of fluid drawn within the syringe barrel).

The materials of construction for each of the plunger and barrel also enhances the drawing of very small increments of liquid pharmaceutical composition into the barrel in very discrete and small volumetric amounts. For example, the combination of forming the plunger from a polymer material comprising, preferably consisting of, polystyrene and forming the barrel from a polymer material comprising, preferably consisting of, polyethylene (e.g., LDPE) provides for a very smooth and gliding interaction of these two components at their frictional and fluid tight interfaces (e.g., between the plunger stopper member 94 and the internal wall surface areas of the barrel first portion 66). This facilitates a smooth and easy withdrawal of the plunger from the barrel in very precise small volume (e.g. less than 0.05 mL) increments when drawing fluid within the fluid chamber of the barrel (as defined at the barrel first portion). Further, the configurations of the adaptor and barrel facilitates case of withdrawal of very small portions remaining in the container during use.

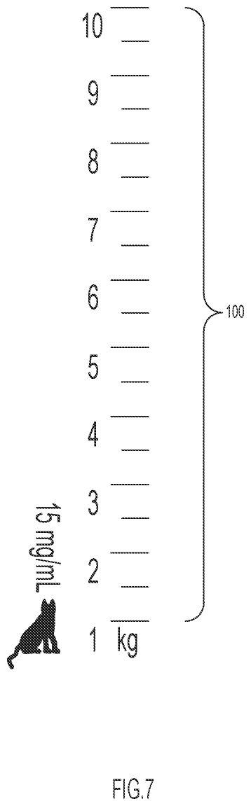

A further enhancement to the syringe 50 includes providing indicia (e.g., printed and/or etched indicia) along an exposed surface wall section of the plunger 80, preferably at the plunger second portion 90, where the indicia comprises a dosage scale 100 of gradation marks comprising lines and/or corresponding numbers (see, e.g., FIG. 7) that are aligned in a direction that corresponds with (e.g., substantially parallel with) the lengthwise dimension of the plunger, where each gradation mark and/or corresponding number is indicative of a specific dosage amount that has been drawn into the barrel forward end 62 (when the barrel 60 is connected with the container 20 in a manner as described herein). Typically, a volumetric or dosage scale of marks for a conventional syringe is provided along the barrel rather than the plunger, where the forward end of the plunger is aligned with a mark of the scale provided on the barrel during drawing of fluid within the barrel (where the barrel is transparent or at least partially translucent) to obtain a desired volume of fluid within the barrel. The syringe 50 as described herein is operable to obtain a precise volume of liquid pharmaceutical composition (and thus precise dosage amount) within the barrel 60 by withdrawing the plunger 80 from the barrel 60 until the desired gradation mark of the dosage scale 100 on the plunger is visible at a designated location along the barrel second portion 70 or on a portion of the plunger second portion 90 that is exposed (i.e., outside of the barrel). Referring to FIG. 6, a precise indication of volume of fluid (i.e., precise dosage amount) within the barrel first portion 66 is determined based upon a gradation mark of the scale 100 along the plunger second portion 90 that is exposed and outside of the barrel 60. In particular, during drawing of the liquid pharmaceutical composition from the container 20, the plunger 80 can be displaced from the barrel 60 until the specific gradation mark from the scale 100 aligns (e.g., is substantially coplanar) with a rear surface 73 (as part of the barrel flange 72) that defines the barrel rear end 64. Alternatively, the plunger can also be aligned with the barrel in any other manner such that a specific gradation mark from the dosage scale aligns with any other portion of the syringe. In another example embodiment, the barrel can include a window or other indicator (e.g., a cut-out portion located along the barrel second portion and/or printed indicia along the barrel second portion) that provides a location at which a specific gradation mark of the dosage scale provided on a surface wall section of the plunger second portion is aligned to indicate the precise volumetric/dosage amount has been withdrawn within the barrel at the barrel first portion. The dosage scale is provided along the plunger second portion at a precise location such that displacement of the plunger from the barrel that aligns a specific gradation mark with an indicator location along the barrel establishes a precise volume of fluid drawn within the barrel first portion.

The gradation marks on the dosage scale 100 for the plunger 80 can be provided in volumetric amounts (e.g., in milliliter or mL amounts) or, alternatively, in any other numeric marker that renders case of use of the syringe in administering a precise dosage to the patient. In an example embodiment (as shown in FIG. 7), a dosage scale 100 for the plunger 80 can include gradation marks that are numerically ordered based upon a body mass or weight of a patient (e.g., feline), where the body mass or weight can be provided in any suitable types of units, such as SI units or in kg (kilograms), or Imperial units or in lb (pounds). This enhances the simplicity or case of the delivery system providing the proper dosage to the patient based upon the patient's weight and the concentration of SGLT-2 inhibitor(s) (and/or other API) in the liquid pharmaceutical composition being used. In particular, this minimizes the potential for user errors in dosage amount by converting the dosage scale to the bodyweight of the patient, which makes this particularly convenient for end users who may not be a health care or medical professional (e.g., veterinarian) but instead the caretaker for the patient (e.g., pet owner for feline patient or other animal patient).

For example, when a required dosage of SGLT-2 inhibitor(s) to a feline patient for treatment of a particular metabolic disorder is 0.1 mg/kg BW, the dosage scale 100 provides an easy conversion for the user for administering the correct volumetric amount from the syringe 50 by simply drawing liquid pharmaceutical composition from the container 20 into the syringe barrel 60 by displacing the plunger 80 from the barrel 60 until the gradation mark on the scale 100 that represents the feline patient's weight in kilograms (kg) is aligned with a corresponding indicator of the syringe (e.g., gradation mark on plunger is aligned or substantially coplanar with the rear surface 73 of the barrel flange 72). It is noted that such markings in bodyweight of the patient will require a dosage scale that corresponds with SGLT-2 inhibitor (and/or other API) concentration in the liquid pharmaceutical composition being administered (e.g., 15 mg/mL as shown in FIG. 7). The previously noted key-like connection between syringe and container (via the adaptor for the container) ensures that the appropriate syringe with dosage scale corresponds with the correct liquid pharmaceutical composition that is being withdrawn from the container.

As previously noted, the liquid pharmaceutical composition including in particular one or more SGLT-2 inhibitor compound(s) and delivery system (container with syringe) can be combined as a single kit-in-parts and made available for commercial or other end use together by providing in a single package as a kit-in-parts. The kit-in-parts can include a single packaged structure that includes container with liquid pharmaceutical composition and syringe. Each of these components can be sub-packaged (i.e., provided in a separate package) within the single kit-in-parts package. The kit-in-parts can further include suitable instructions, e.g., provided in a package insert or leaflet, that includes information on administration of the liquid pharmaceutical composition and use of the delivery system for treatment and/or prevention of a metabolic disorder and/or other medical indication as described herein for a specified patient.

The delivery system can be configured for administration of the liquid pharmaceutical composition to animal patients such as equines, ruminants, canines, and felines. The administration route can be oral or parenteral, but is preferably oral. The delivery system 10 as described herein includes a needle-less syringe, where liquid pharmaceutical composition is drawn within the syringe barrel 60 via engagement of the barrel forward end 62 with the adaptor 40 of the of the container 20. However, it is noted that the delivery system can also include a needle for parenteral administration for certain embodiments.

An example embodiment is now described demonstrating operation of the delivery system 10 for treatment and/or prevention of a metabolic disorder or another medical indication in a patient by administering a liquid pharmaceutical composition including in particular one or more SGLT-2 inhibitor(s) to the patient. In this example embodiment, the patient is a feline, and the dosage scale 100 for the syringe 80 is as presented in FIG. 7 (gradation marks for the scale 100 are in relation to bodyweight in kg for the feline).

Prior to engagement of the syringe 50 with the container 20, the plunger 80 is fully inserted within the barrel 60 such that the plunger forward end 82 including stopper member 94 are in contact with the interior surface at the barrel forward end 62. After removal of the cap 30 from the container 20, the syringe 50 (including plunger 80 installed within the barrel 60 as shown, e.g., in FIGS. 1 and 6) is connected with the container 20 by inserting the barrel forward end 62 into the inner connection member 48 of the adaptor 40 sufficiently to ensure a frictional and fluid tight engagement. Next, the container 20 with engaged syringe 50 can be inverted (e.g., vertically oriented so that the container upper end 26 is below the container lower end 27 and the syringe 50 is locater under the container 20). This results in the liquid pharmaceutical composition being disposed within the container 20 at its upper end 26 substantially without any air gap or void at the interface between the interior surface portions of the adaptor 40 within the container 20 and liquid pharmaceutical composition in contact with such interior surface portions. Further, and as can be seen, e.g., in FIGS. 5 and 6, the gradation marks of the dosage scale 100 can be inverted (e.g., upside down) in relation to the forward end 82 of the plunger 80. This facilitates case of reading the dosage scale 100 (e.g., gradation marks will be right side up) when the syringe 50 is inverted and liquid pharmaceutical composition is drawn into the barrel 60.

The plunger rear end 92 and plunger forward end 82 are displaced a selected distance from the respective barrel rear end 72 and barrel forward end 62 until a specified gradation mark along dosage scale 100 on the plunger second portion 90 that corresponds with the feline patient's weight (in kg) is suitably aligned (e.g., coplanar) with the rear surface 73 of the barrel flange 72. For example, if the feline weight is 4 kg, the plunger 80 is displaced from the barrel until gradation mark for the number 4 on the dosage scale 100 is aligned (e.g., coplanar) with the barrel flange surface 73 (e.g., as depicted in FIG. 6). This displacement of the plunger 80 from the barrel 60 results in withdrawal of liquid pharmaceutical composition from the container 20, through the inner connection member 48 of the adaptor 40, and through the opening at the end wall 74 of the barrel forward end 62 into the barrel first portion 66. The displacement of the plunger 80 from the barrel 60 so as to align the specified gradation mark from the scale 100 with the barrel flange rear surface 73 ensures that the precise dosage amount of liquid pharmaceutical composition has been drawn from the container 20 into the barrel first portion 66. The configuration of plunger with barrel, as well as frictional and fluid tight fit between the barrel forward end 66 and the inner connection member 48 of the adaptor 40, ensures that a sufficient vacuum force is generated within the barrel first portion 66 when the plunger rear end 84 is displaced a selected distance from the barrel rear end 64, which results in substantially the entire volume defined by the interior of the barrel first portion 66 between the barrel forward end 62 and the stopper member 94 of the syringe 80 corresponds with the volume of liquid pharmaceutical composition drawn within the barrel.

The specified dosage amount of liquid pharmaceutical composition drawn within the syringe barrel of the delivery system can be administered orally to the feline patient directly by injecting the composition from the barrel 60 (i.e., displacing the plunger 80 back into the barrel 60 to force the composition through the opening in the end wall 74 at the barrel forward end 62) into the feline patient's mouth. Alternatively, the dosage amount can be indirectly administered orally by providing the liquid pharmaceutical composition from the syringe 50 to a liquid (e.g., water) or food for consumption by the feline patient. The package insert or leaflet provide information to the end user with regard to daily dosage amounts (e.g., number of doses per day) to be provided to the feline patient based upon the particulars of the metabolic disorder or other medical indication which is the subject of treatment.

Thus, the embodiments described herein facilitate the administration of a liquid pharmaceutical composition including in particular one or more SGLT-2 inhibitor compound(s) in very small, discrete dosage amounts for the treatment and/or prevention of one or more metabolic disorders and/or one or more other medical indications. The delivery systems with key-like features can ensure that the proper syringe (with dosage indications specific to a specific composition, specific patient type and/or specific medical treatment) is matched with proper container with specific composition for administration to a patient.

In particular, example embodiments of the invention have been described herein as follows by means of the following clauses, which are also part of the disclosure and are comprised by the spirit and scope of the present invention:

1. A system for administering a liquid pharmaceutical composition to a patient, the system comprising a syringe, wherein the syringe comprises:
a barrel comprising a hollow elongated member including an open barrel forward end and an open barrel rear end, the hollow elongated member comprising:
a first portion that extends from the open barrel forward end to a transition section disposed along the hollow elongated member, the first portion having a first cross-sectional dimension; and
a second portion that extends from the transition section to the open barrel rear end, the second portion having a second cross-sectional dimension that is greater than the first cross-sectional dimension; and
a plunger comprising an elongated member including a plunger forward end and a plunger rear end;
wherein:
the plunger and the barrel are dimensioned such that a portion of the plunger including the plunger forward end is insertable within the hollow elongated member of the barrel at the open barrel rear end, and the plunger is extendible within the barrel until the plunger forward end engages with an internal surface portion of the hollow elongated member at the barrel forward end;
receipt of liquid pharmaceutical composition within the barrel of the syringe via the open barrel forward end is limited to a volume defined within the first portion of the barrel, wherein the volume is adjustable by adjusting a displacement of the plunger forward end away from the barrel forward end; and
a surface wall section of the plunger includes indicia comprising a dosage scale that indicates the volume of the liquid pharmaceutical composition received within the first portion of the barrel based upon a corresponding displacement of the plunger forward end away from the barrel forward end.

2. The system according to clause 1, wherein the barrel is formed of a polymer material comprising, preferably consisting of, polyethylene, most preferably consisting of low density polyethylene, and the plunger is formed of a polymer material comprising, preferably consisting of, polystyrene.

3. The system according to any one of clauses 1 to 2, wherein the second portion of the barrel has a length to diameter (L/D) ratio from 5 to 20, or from 7 to 15, or from 9 to 14.

4. The system according to any one of clauses 1 to 3, wherein the second portion of the barrel defines a volume within the hollow elongated member that is no greater than 1 mL, or no greater than 0.9 mL, or no greater than 0.8 mL, or no greater than 0.7 mL, or no greater than 0.6 mL, or no greater than 0.5 mL, or no greater than 0.4 mL.

5. The system according to any one of clauses 1 to 4, wherein:
the dosage scale on the surface wall section of the plunger comprises a plurality of marks aligned in a direction that corresponds with a lengthwise dimension of the plunger; and
in operation, the volume of the liquid pharmaceutical composition received within the first portion of the barrel in response to displacement of the plunger forward end away from the barrel forward end is indicated by alignment of a corresponding mark of the dosage scale with an exterior surface of the barrel at the barrel rear end.

6. The system according to any one of clauses 1 to 5, further comprising:
a container that contains the liquid pharmaceutical composition.

7. The system according to clause 6, wherein the liquid pharmaceutical composition comprises one or more SGLT-2 inhibitor compound(s) selected from the group consisting of:
(1) a glucopyranosyl-substituted benzene derivative represented by the following formula:

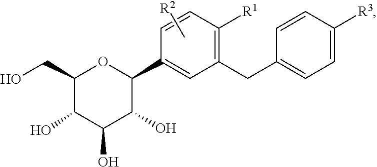

wherein R₁ denotes cyano, Cl or methyl (most preferably cyano),
R₂ denotes H, methyl, methoxy or hydroxy (most preferably H), and
R₃ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R₃ is preferably selected from cyclopropyl, ethyl, ethinyl, ethoxy, (R)-tetrahydrofuran-3-yloxy or (S)-tetrahydrofuran-3-yloxy; and most preferably R₃ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by the following formula:

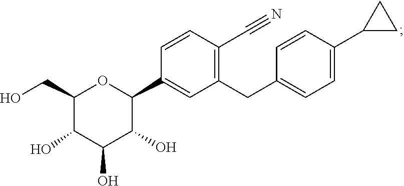 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by the following formula:

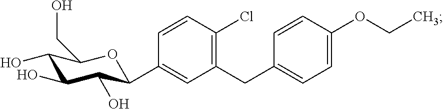

(4) Canagliflozin, represented by the following formula:

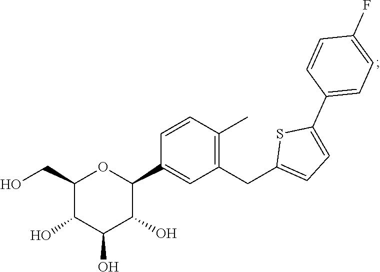 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by the following formula:

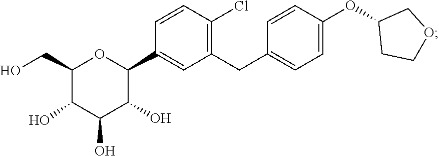 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luseogliflozin, represented by the following formula:

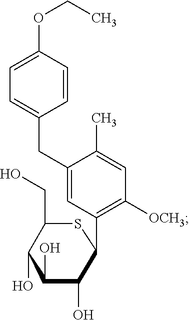

(7) Tofogliflozin, represented by the following formula:

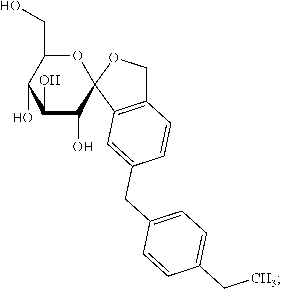

(8) Ipragliflozin, represented by the following formula:

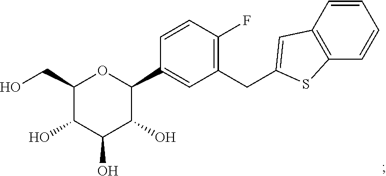 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by the following formula:

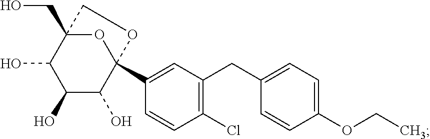 [SMILES: CCOc1ccc(Cc2cc([C@]34OC[C@](CO)(O3)[C@@H](O)[C@H](O)[C@H]4O)ccc2Cl)cc1]

(10) Atigliflozin, represented by the following formula:

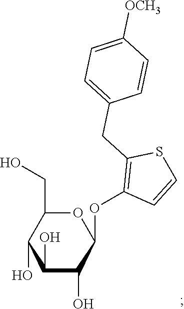

(11) Remogliflozin, represented by the following formula:

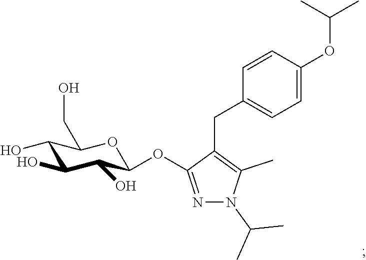

(11A) Remogliflozin etabonate, represented by the following formula:

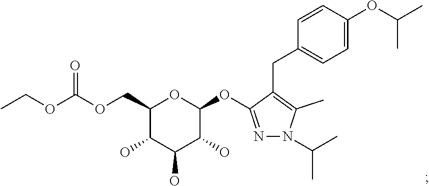 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative represented by the following formula:

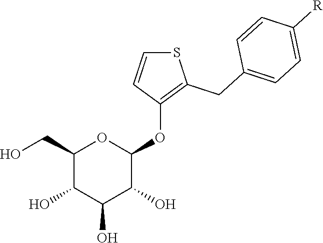

wherein R denotes methoxy or trifluoromethoxy;
(13) 1-(β-D-glucopyranosyl)-4-methyl-3-[5-(4-fluorophenyl)-2-thienylmethyl]benzene, represented by the following formula:

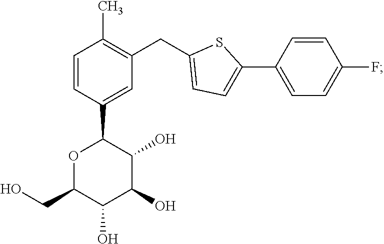 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative represented by the following formula:

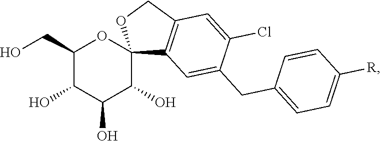

wherein R denotes methoxy, trifluoromethoxy, ethoxy, ethyl, isopropyl or tert. butyl;
(15) a pyrazole-O-glucoside derivative represented by the following formula:

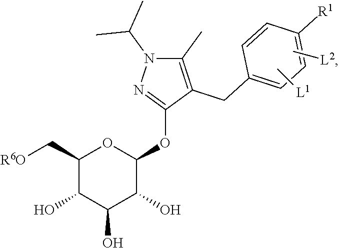

wherein:
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by the following formula:

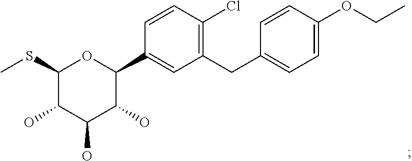 [SMILES: CCOc1ccc(Cc2cc([C@@H]3O[C@H](SC)[C@@H](O)[C@H](O)[C@H]3O)ccc2Cl)cc1]

(17) Sergliflozin, represented by the following formula:

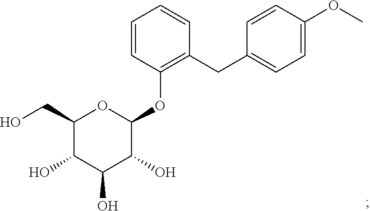 [SMILES: COc1ccc(Cc2ccccc2O[C@@H]2O[C@H](CO)[C@@H](O)[C@H](O)[C@H]2O)cc1]

(18) a compound represented by the following formula:

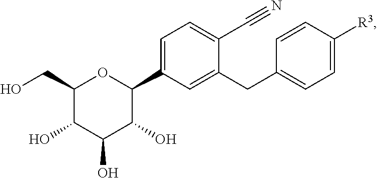

wherein:
R₃ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R₃ is preferably selected from cyclopropyl, ethyl, ethinyl, ethoxy, (R)-tetrahydrofuran-3-yloxy or (S)-tetrahydrofuran-3-yloxy; and R₃ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by the following formula:

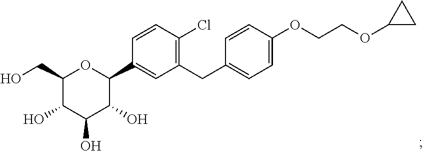 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by the following formula:

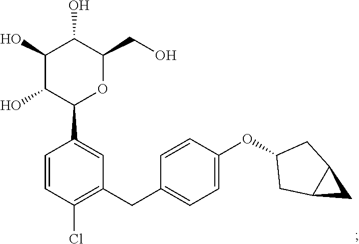 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin;

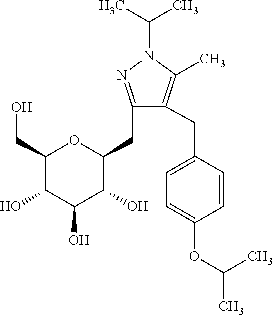 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
(23) Enavogliflozin, represented by the following formula:

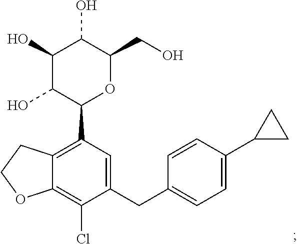 [SMILES: OC[C@H]1O[C@@H](c2cc(Cc3ccc(C4CC4)cc3)c(Cl)c3c2CCO3)[C@H](O)[C@@H](O)[C@@H]1O]

and
(24) TFC-039, represented by the following formula:

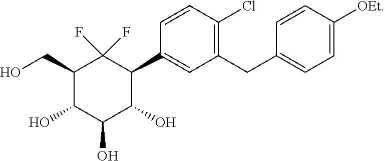 [SMILES: CCOc1ccc(Cc2cc([C@H]3[C@H](O)[C@@H](O)[C@H](O)[C@@H](CO)C3(F)F)ccc2Cl)cc1]

8. The system according to clause 7, wherein the concentration of the one or more SGLT-2 inhibitor compound(s) within the liquid pharmaceutical composition is from 0.1 mg/mL to 20 mg/mL.

9. The system according to clause 8, wherein the one or more SGLT-2 inhibitor compound(s) comprises Velagliflozin, wherein preferably Velagliflozin is the only SGLT-2 inhibitor contained in the liquid pharmaceutical composition.

10. The system according to clause 9, wherein the concentration of Velagliflozin within the liquid pharmaceutical composition is 1.2 mg/mL or 15 mg/mL.

11. The system according to any one of clauses 1 to 10, further comprising:
an adaptor connected with an opening in the container, the adaptor comprising a hollow connection member that facilitates coupling of the barrel forward end with the connection member to facilitate transfer of the liquid pharmaceutical composition to the first portion of the barrel, wherein a portion of the hollow connection member of the adaptor has an outer diameter that is substantially similar to an inner diameter at the opening in the container to establish a frictional and fluid tight connection between the adaptor and the container, and the hollow connection member of the adaptor has an inner diameter that is substantially similar to an outer diameter of the barrel forward end to establish a frictional and fluid tight connection between the adaptor and the barrel in response to insertion of the barrel forward end into the hollow connection member.

12. The system according to any one of clauses 1 to 11, wherein the adaptor is formed of a polymer material comprising, preferably consisting of, low density polyethylene, and the container is formed of a polymer material comprising, preferably consisting of, high density polyethylene.

13. One or more SGLT-2 inhibitor compound(s) for use in a method of treatment and/or prevention of a metabolic disorder and/or another medical condition of a patient by administering a dosage of a liquid pharmaceutical composition comprising one or more SGLT-2 inhibitor compound(s) to a patient, the method comprising:
providing a syringe and a container that includes the liquid pharmaceutical composition, the syringe comprising a barrel and a plunger, wherein:
the barrel includes a hollow elongated member with an open barrel forward end and an open barrel rear end, a first portion that extends from the open barrel forward end to a transition section disposed along the hollow elongated member, the first portion having a first cross-sectional dimension, and a second portion that extends from the transition section to the open barrel rear end, the second portion having a second cross-sectional dimension that is greater than the first cross-sectional dimension;
the plunger includes an elongated member and a dosage scale, the elongated member including a plunger forward end and a plunger rear end, and the dosage scale comprises a plurality of marks disposed on a surface wall section of the plunger and aligned in a direction that corresponds with a lengthwise dimension of the plunger; and
the plunger and the barrel are dimensioned such that a portion of the plunger including the plunger forward end is insertable within the hollow elongated member of the barrel at the open barrel rear end, and the plunger is extendible within the barrel until the plunger forward end engages with an internal surface portion of the hollow elongated member at the barrel forward end;
withdrawing a desired dosage amount of the liquid pharmaceutical composition from the container into the first portion of the barrel of the syringe by displacing the plunger forward end a selected distance from the barrel forward end until a selected mark of the plurality of marks for the dosage scale aligns with an exterior surface of the barrel at the barrel rear end; and
administering the desired dosage amount of the liquid pharmaceutical composition from the syringe to the patient.
(A corresponding method of treatment and/or prevention of a metabolic disorder and/or another medical condition of a patient by administering a dosage of a liquid pharmaceutical composition comprising one or more SGLT-2 inhibitor compound(s) to a patient, as well as the corresponding use of one or more SGLT-2 inhibitor compound(s) for the preparation of a medicament for the treatment and/or prevention of a metabolic disorder and/or another medical condition of a patient, are also intended to be comprised by the present invention.)

14. The one or more SGLT-2 inhibitor compound(s) for use in a method according to clause 13, wherein the administering the desired dosage amount of the liquid pharmaceutical composition from the syringe to the patient comprises oral administration or parenteral administration to the patient, preferably oral administration.

15. The one or more SGLT-2 inhibitor compound(s) for use in a method according to any one of clauses 13 to 14, wherein the administering the desired dosage amount of the liquid pharmaceutical composition from the syringe to the patient comprises oral administration to the patient by direct transfer of the liquid pharmaceutical composition from the syringe orally to the patient or by indirect transfer of the liquid pharmaceutical composition from the syringe to food or liquid for consumption by the patient.

16. The one or more SGLT-2 inhibitor compound(s) for use in a method according to any one of clauses 13 to 15, wherein the desired dosage amount is no greater than 1 mL, or no greater than 0.9 mL, or no greater than 0.8 mL, or no greater than 0.7 mL, or no greater than 0.6 mL, or no greater than 0.5 mL, or no greater than 0.4 mL.

17. The one or more SGLT-2 inhibitor compound(s) for use in a method according to any one of clauses 13 to 16, wherein the desired dosage amount ranges from 0.05 mL to 0.6 mL.

18. The one or more SGLT-2 inhibitor compound(s) for use in a method according to any one of clauses 13 to 17, wherein the one or more SGLT-2 inhibitor compound(s) is selected from the group consisting of:
(1) a glucopyranosyl-substituted benzene derivative represented by the following formula:

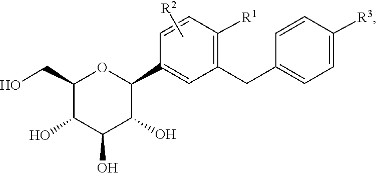

wherein R₁ denotes cyano, Cl or methyl (most preferably cyano),
R₂ denotes H, methyl, methoxy or hydroxy (most preferably H), and
R₃ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R₃ is preferably selected from cyclopropyl, ethyl, ethinyl, ethoxy, (R)-tetrahydrofuran-3-yloxy or (S)-tetrahydrofuran-3-yloxy; and most preferably R₃ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by the following formula:

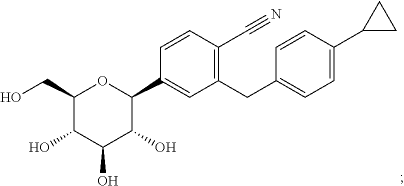 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by the following formula:

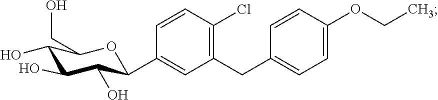

(4) Canagliflozin, represented by the following formula:

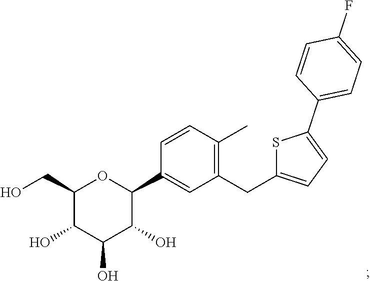 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by the following formula:

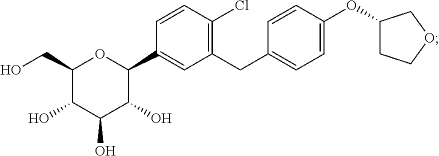 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luscogliflozin, represented by the following formula:

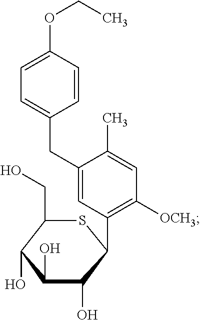

(7) Tofogliflozin, represented by the following formula:

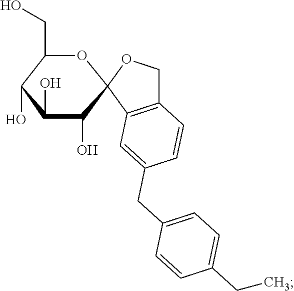

(8) Ipragliflozin, represented by the following formula:

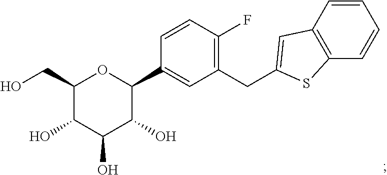 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by the following formula:

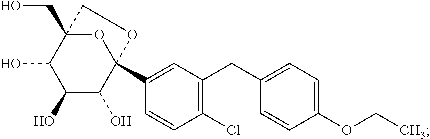 [SMILES: CCOc1ccc(Cc2cc([C@]34OC[C@](CO)(O3)[C@@H](O)[C@H](O)[C@H]4O)ccc2Cl)cc1]

(10) Atigliflozin, represented by the following formula:

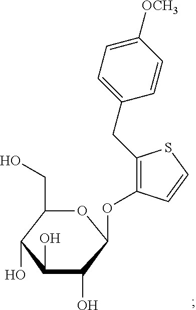

(11) Remogliflozin, represented by the following formula:

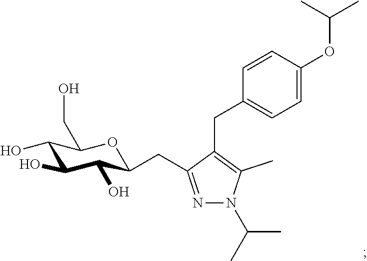

(11A) Remogliflozin etabonate, represented by the following formula:

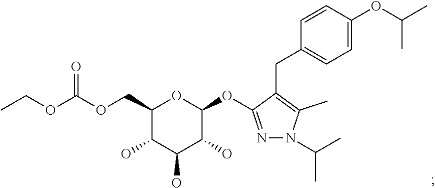 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative represented by the following formula:

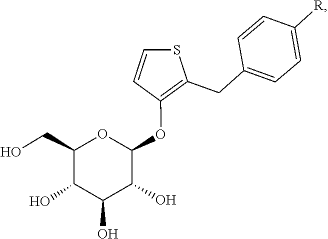

wherein R denotes methoxy or trifluoromethoxy;
(13) 1-(β-D-glucopyranosyl)-4-methyl-3-[5-(4-fluorophenyl)-2-thienylmethyl]benzene, represented by the following formula:

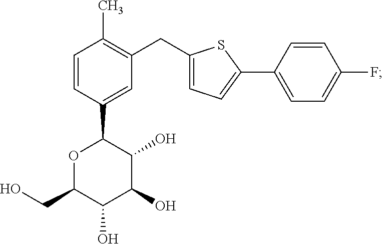 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative represented by the following formula:

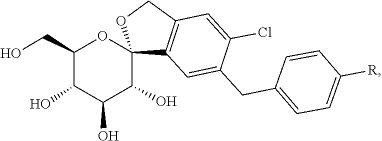

wherein R denotes methoxy, trifluoromethoxy, ethoxy, ethyl, isopropyl or tert. butyl;
(15) a pyrazole-O-glucoside derivative represented by the following formula:

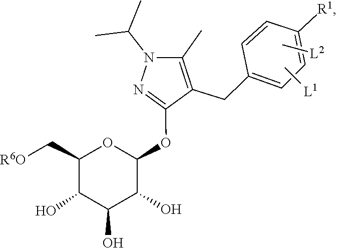

wherein:
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by the following formula:

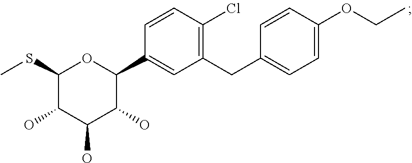 [SMILES: CCOc1ccc(Cc2cc([C@@H]3O[C@H](SC)[C@@H](O)[C@H](O)[C@H]3O)ccc2Cl)cc1]

(17) Sergliflozin, represented by the following formula:

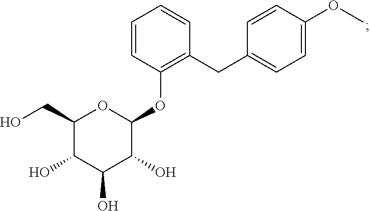 [SMILES: COc1ccc(Cc2ccccc2O[C@@H]2O[C@H](CO)[C@@H](O)[C@H](O)[C@H]2O)cc1]

(18) a compound represented by the following formula:

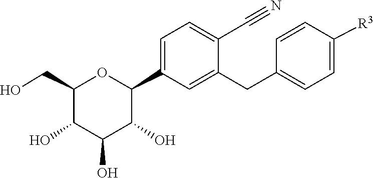

wherein:
R₃ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R₃ is preferably selected from cyclopropyl, ethyl, ethinyl, ethoxy, (R)-tetrahydrofuran-3-yloxy or (S)-tetrahydrofuran-3-yloxy; and R₃ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by the following formula:

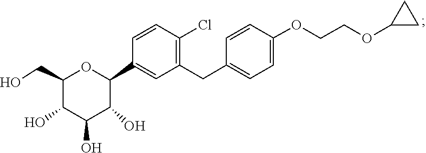 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by the following formula:

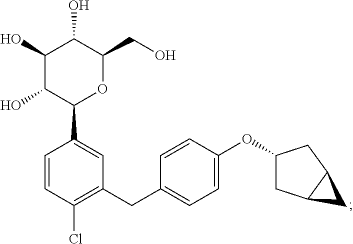 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin;

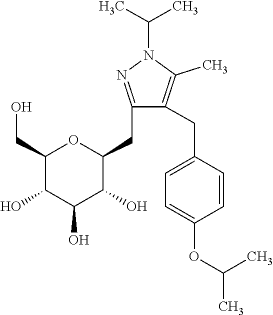 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
(23) Enavogliflozin, represented by the following formula:

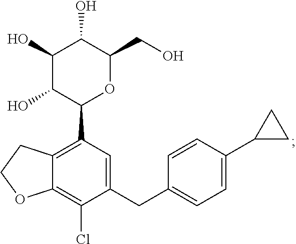 [SMILES: OC[C@H]1O[C@@H](c2cc(Cc3ccc(C4CC4)cc3)c(Cl)c3c2CCO3)[C@H](O)[C@@H](O)[C@@H]1O]

and
(24) TFC-039, represented by the following formula:

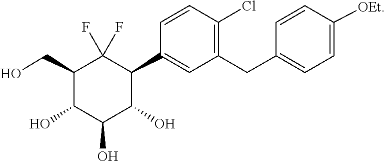 [SMILES: CCOc1ccc(Cc2cc([C@H]3[C@H](O)[C@@H](O)[C@H](O)[C@@H](CO)C3(F)F)ccc2Cl)cc1]

19. The one or more SGLT-2 inhibitor compound(s) for use in a method according to clause 18, wherein the one or more SGLT-2 inhibitor compound(s) comprises Velagliflozin, preferably wherein Velagliflozin is the only SGLT-2 inhibitor compound administered.

20. The one or more SGLT-2 inhibitor compound(s) for use in a method according to any one of clauses 13 to 19, wherein the dosage amount is from 0.01 to 10 mg/kg bodyweight of the patient.

21. The one or more SGLT-2 inhibitor compound(s) for use in a method according to any one of clauses 13 to 20, wherein the patient is a non-human mammal selected from the group of equines, ruminants, felines and canines, preferably a feline or a (small) canine.

22. The one or more SGLT-2 inhibitor compound(s) for use in a method according to any one of clauses 13 to 21, wherein the metabolic disorder and/or other medical condition is selected from the group consisting of:
(i) a metabolic disorder of an equine animal, wherein preferably the metabolic disorder is one or more disorders selected from insulin resistance, hyperinsulinemia, impaired glucose tolerance, dyslipidemia, dysadipokinemia, subclinical inflammation, systemic inflammation, low grade systemic inflammation, obesity, and/or regional adiposity, wherein preferably the metabolic disorder is insulin resistance, hyperinsulinemia, and/or a clinical condition associated with insulin resistance and/or hyperinsulinemia; wherein preferably said clinical condition is one or more conditions selected from impaired glucose tolerance, dyslipidemia, dysadipokinemia, subclinical inflammation, systemic inflammation, low grade systemic inflammation, obesity, and/or regional adiposity;
(ii) a metabolic disorder of an equine animal, wherein the metabolic disorder is one or more disorders selected from laminitis, vascular dysfunction, hypertension, hepatic lipidosis, atherosclerosis, hyperadrenocorticism, Pituitary Pars Intermedia Dysfunction and/or Equine Metabolic Syndrome, wherein preferably the metabolic disorder is a clinical condition/sign associated with insulin resistance and/or hyperinsulinemia, wherein said clinical condition/sign preferably is one or more conditions selected from laminitis, vascular dysfunction, hypertension, hepatic lipidosis, atherosclerosis, hyperadrenocorticism, Pituitary Pars Intermedia Dysfunction and/or Equine Metabolic Syndrome;
(iii) a metabolic disorder of a feline animal, wherein preferably the metabolic disorder is one or more selected from the group consisting of: ketoacidosis, pre-diabetes, diabetes mellitus type 1 or type 2, insulin resistance, acromegaly, diabetes with elevated IGF-1 concentration, obesity, hyperglycemia, impaired glucose tolerance, hyperinsulinemia, dyslipidemia, dysadipokinemia, subclinical inflammation, systemic inflammation, low grade systemic inflammation, hepatic lipidosis, atherosclerosis, inflammation of the pancreas, neuropathy and/or Syndrome X (metabolic syndrome) and/or loss of pancreatic beta cell function and/or wherein the remission of the metabolic disorder, preferably diabetic remission, is achieved and/or maintained;
(iv) a metabolic disorder of a canine animal, wherein preferably the metabolic disorder is one or more selected from the group consisting of: ketoacidosis, pre-diabetes, insulin dependent diabetes mellitus, insulin resistance diabetes, insulin resistance, obesity, hyperglycemia, hyperglycemia induced cataract formation, impaired glucose tolerance, hyperinsulinemia, dyslipidemia, dysadipokinemia, subclinical inflammation, systemic inflammation, low grade systemic inflammation, hepatic lipidosis, inflammation of the pancreas, metabolic disorder consequences, such as hypertension, renal dysfunction and/or musculoskeletal disorders, and/or Syndrome X (metabolic syndrome), preferably pre-diabetes, insulin dependent diabetes mellitus, insulin resistance diabetes, insulin resistance, wherein preferably the development of hyperglycemia induced cataract formation is prevented or remission is achieved and/or wherein preferably the development of metabolic disorder consequences, such as hypertension, renal dysfunction and/or musculoskeletal disorders, is prevented or progression is slowed or remission is achieved;
(v) a cardiac disease of a feline animal, wherein preferably the cardiac disease is one or more selected from the group consisting of: heart failure, heart failure due to one or more cardiomyopathies, heart failure due to hypertrophic cardiomyopathy (HCM), heart failure due to restrictive cardiomyopathy (RCM), heart failure due to dilated cardiomyopathy (DCM), heart failure due to unclassified cardiomyopathy (UCM), heart failure due to arrhythmogenic right ventricular cardiomyopathy (ARVC), hypertrophic cardiomyopathy (HCM), restrictive cardiomyopathy (RCM), dilated cardiomyopathy (DCM), unclassified cardiomyopathy (UCM), and/or arrhythmogenic right ventricular cardiomyopathy (ARVC); preferably selected from the group consisting of: heart failure due to one or more cardiomyopathies, heart failure due to hypertrophic cardiomyopathy (HCM), hypertrophic cardiomyopathy (HCM);
(vi) drying-off of a non-human mammal, preferably ruminant, wherein preferably the medical condition is one or more selected from the group consisting of: improving and/or facilitating the drying-off of a non-human mammal, preferably ruminant, reducing the milk production, preferably milk production and/or secretion, in a pregnant and/or lactating non-human mammal, preferably ruminant, decreasing milk accumulation and/or engorgement in the udder, preferably udder and/or mammary gland, of a non-human mammal, preferably ruminant, decreasing the discomfort associated with udder engorgement, such as increasing the daily lying time and/or reduction of stress, of a non-human mammal, preferably ruminant, decreasing milk leakage after drying-off of a non-human mammal, preferably ruminant, decreasing the incidence of intra-mammary infections (IMI), preferably mastitis and/or metritis, in a non-human mammal, preferably ruminant;
(vii) a cardiac disease of a non-human mammal, excluding a feline, in particular a canine, wherein preferably the cardiac disease is one or more selected from the group consisting of: heart failure; congestive heart failure; asymptomatic/preclinical/occult heart failure; heart failure due to (myxomatous) mitral valve disease [(M)MVD]; congestive heart failure due to (myxomatous) mitral valve disease [(M)MVD]; asymptomatic/preclinical/occult heart failure due to (myxomatous) mitral valve disease [(M)MVD]; (myxomatous) mitral valve disease [(M)MVD]; clinically overt (myxomatous) mitral valve disease [(M)MVD]; asymptomatic/preclinical/occult (myxomatous) mitral valve disease [(M)MVD]; heart failure due to dilated cardiomyopathy (DCM); congestive heart failure due to dilated cardiomyopathy (DCM); asymptomatic/preclinical/occult heart failure due to dilated cardiomyopathy (DCM); dilated cardiomyopathy (DCM); clinically overt dilated cardiomyopathy (DCM); asymptomatic/preclinical/occult dilated cardiomyopathy (DCM); aortic stenosis (valvular, supravalvular and/or subvalvular);
(viii) hypertension in a non-human mammal, preferably a carnivore, more preferably a cat or a dog, wherein preferably the hypertension is one or more selected from the group consisting of: situational hypertension, secondary hypertension and idiopathic hypertension, wherein preferably the secondary hypertension is selected from the group consisting of hypertension associated with chronic kidney disease (CKD), diabetes, obesity, heart disease, endocrine disease, such as Cushing's disease, hyperthyroidism, acromegaly, and elevated blood pressure (BP) induced by medicaments, preferably by glucocorticoids, mineralocorticoids, erythropoiesis-stimulating agents, ephedrine and/or high dose sodium chloride;
(ix) a renal disease of a non-human mammal, preferably a carnivore, more preferably a cat or a dog, wherein preferably the renal disease is one or more selected from the group consisting of: renal dysplasia, glomerulopathy, polycystic kidney disease, amyloidosis, tubulo-nephritis/tubulointerstitial nephritis (TIN), acute kidney disease, chronic kidney disease.

It is intended that the present invention covers the modifications and variations of this invention provided they come within the scope of the appended claims and their equivalents. It is to be understood that relative terms such as “top”, “bottom”, “forward”, “rear”, “side”, “height”, “length”, “width”, “upper”, “lower”, “interior”, “exterior”, and the like as may be used herein, merely describe points of reference and do not limit the present invention to any particular orientation or configuration.

What is claimed:

1. A system for administering a liquid pharmaceutical composition to a patient, the system comprising a syringe, wherein the syringe comprises:
a barrel comprising a hollow elongated member including an open barrel forward end and an open barrel rear end, the hollow elongated member comprising:
a first portion that extends from the open barrel forward end to a transition section disposed along the hollow elongated member, the first portion having a first cross-sectional dimension; and
a second portion that extends from the transition section to the open barrel rear end, the second portion having a second cross-sectional dimension that is greater than the first cross-sectional dimension; and
a plunger comprising an elongated member including a plunger forward end and a plunger rear end;
wherein:
the plunger and the barrel are dimensioned such that a portion of the plunger including the plunger forward end is insertable within the hollow elongated member of the barrel at the open barrel rear end, and the plunger is extendible within the barrel until the plunger forward end engages with an internal surface portion of the hollow elongated member at the barrel forward end;
receipt of liquid pharmaceutical composition within the barrel of the syringe via the open barrel forward end is limited to a volume defined within the first portion of the barrel, wherein the volume is adjustable by adjusting a displacement of the plunger forward end away from the barrel forward end; and
a surface wall section of the plunger includes indicia comprising a dosage scale that indicates the volume of the liquid pharmaceutical composition received within the first portion of the barrel based upon a corresponding displacement of the plunger forward end away from the barrel forward end.

2. The system according to claim 1, wherein the barrel is formed of a polymer material comprising, preferably consisting of, polyethylene, most preferably consisting of low density polyethylene, and the plunger is formed of a polymer material comprising, preferably consisting of, polystyrene.

3. The system according to claim 1, wherein the first portion of the barrel has a length to diameter (L/D) ratio from 5 to 20, or from 7 to 15, or from 9 to 14.

4. The system according to claim 1, wherein the first portion of the barrel defines a volume within the hollow elongated member that is no greater than 3 mL, or no greater than 2 mL, or no greater than 1 mL, or no greater than 0.9 mL, or no greater than 0.8 mL, or no greater than 0.7 mL, or no greater than 0.6 mL, or no greater than 0.5 mL, or no greater than 0.4 mL.

5. The system according to claim 1, wherein:
the dosage scale on the surface wall section of the plunger comprises a plurality of marks aligned in a direction that corresponds with a lengthwise dimension of the plunger; and
in operation, the volume of the liquid pharmaceutical composition received within the first portion of the barrel in response to displacement of the plunger forward end away from the barrel forward end is indicated by alignment of a corresponding mark of the dosage scale with an exterior surface of the barrel at the barrel rear end.

6. The system according to claim 1, further comprising:
a container that contains the liquid pharmaceutical composition.

7. The system according to claim 6, wherein the liquid pharmaceutical composition comprises one or more SGLT-2 inhibitor compound(s) selected from the group consisting of:
(1) a glucopyranosyl-substituted benzene derivative represented by the following formula: 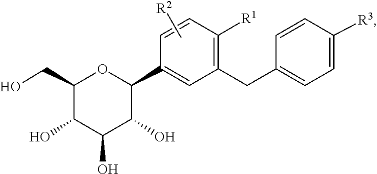
wherein R¹ denotes cyano, Cl or methyl (most preferably cyano),
R² denotes H, methyl, methoxy or hydroxy (most preferably H), and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by the following formula: 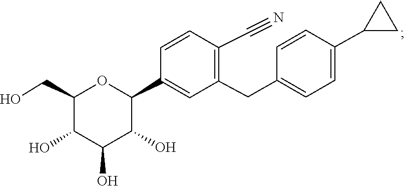 [SMILES: N#Cc1ccc([C@@H]2O[C@H](CO)[C@@H](O)[C@H](O)[C@H]2O)cc1Cc1ccc(C2CC2)cc1]
(3) Dapagliflozin, represented by the following formula: 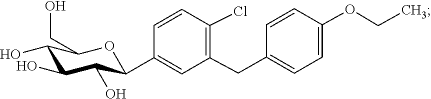
(4) Canagliflozin, represented by the following formula: 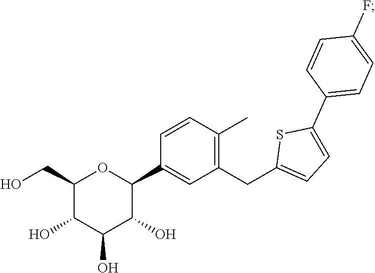 [SMILES: Cc1ccc([C@@H]2O[C@H](CO)[C@@H](O)[C@H](O)[C@H]2O)cc1Cc1ccc(-c2ccc(F)cc2)s1]
(5) Empagliflozin, represented by the following formula: 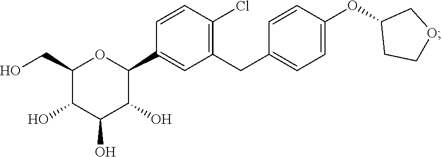 [SMILES: OC[C@H]1O[C@@H](c2ccc(Cl)c(Cc3ccc(O[C@H]4CCOC4)cc3)c2)[C@H](O)[C@@H](O)[C@@H]1O]
(6) Luseogliflozin, represented by the following formula: 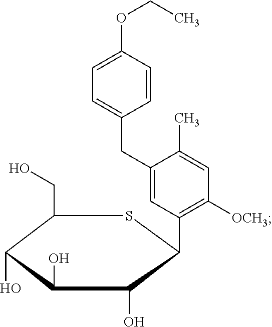
(7) Tofogliflozin, represented by the following formula: 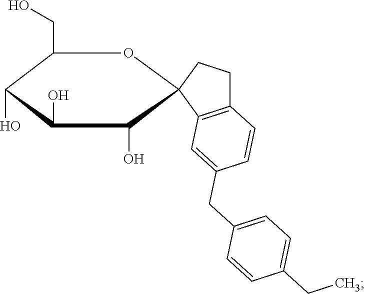
(8) Ipragliflozin, represented by the following formula: 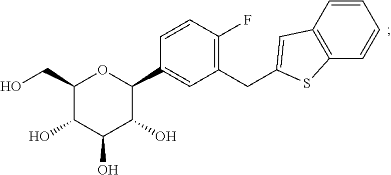 [SMILES: OC[C@H]1O[C@@H](c2ccc(F)c(Cc3cc4ccccc4s3)c2)[C@H](O)[C@@H](O)[C@@H]1O]
(9) Ertugliflozin, represented by the following formula: 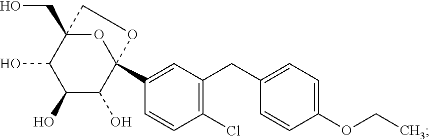 [SMILES: CCOc1ccc(Cc2cc([C@]34OC[C@](CO)(O3)[C@@H](O)[C@H](O)[C@H]4O)ccc2Cl)cc1]
(10) Atigliflozin, represented by the following formula: 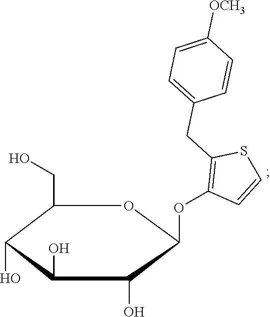
(11) Remogliflozin, represented by the following formula: 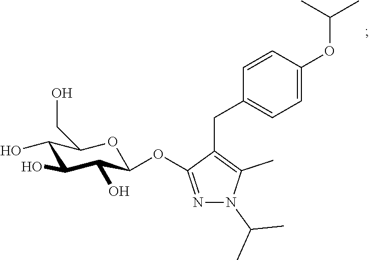
(11A) Remogliflozin etabonate, represented by the following formula: 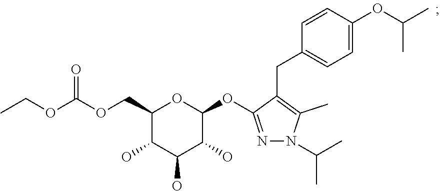 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]
(12) a thiophene derivative represented by the following formula: 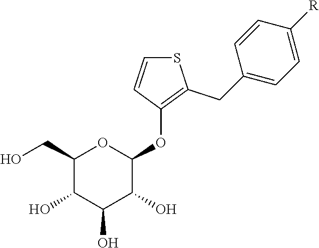
wherein R denotes methoxy or trifluoromethoxy;
(13) 1-(β-D-glucopyranosyl)-4-methyl-3-[5-(4-fluorophenyl)-2-thienylmethyl]benzene, represented by the following formula: 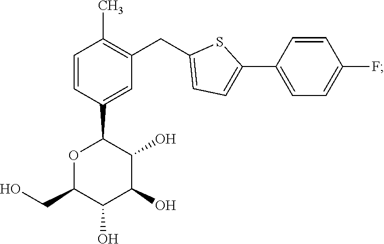 [SMILES: Cc1ccc([C@@H]2O[C@H](CO)[C@@H](O)[C@H](O)[C@H]2O)cc1Cc1ccc(-c2ccc(F)cc2)s1]
(14) a spiroketal derivative represented by the following formula: 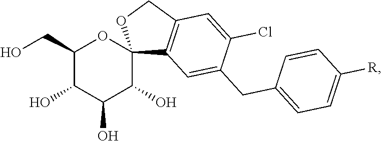
wherein R denotes methoxy, trifluoromethoxy, ethoxy, ethyl, isopropyl or tert. butyl;
(15) a pyrazole-O-glucoside derivative represented by the following formula: 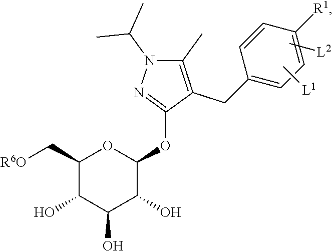
wherein:
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by the following formula: 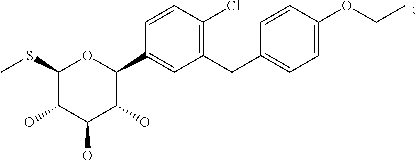 [SMILES: CCOc1ccc(Cc2cc([C@@H]3O[C@H](SC)[C@@H](O)[C@H](O)[C@H]3O)ccc2Cl)cc1]
(17) Sergliflozin, represented by the following formula: 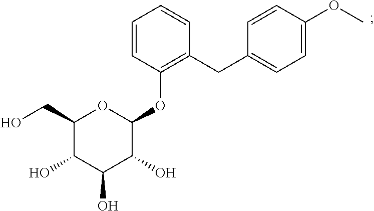 [SMILES: COc1ccc(Cc2ccccc2O[C@@H]2O[C@H](CO)[C@@H](O)[C@H](O)[C@H]2O)cc1]
(18) a compound represented by the following formula: 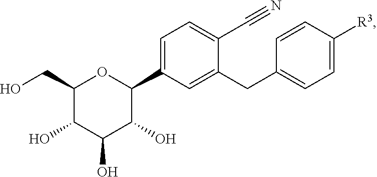
wherein:
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by the following formula: 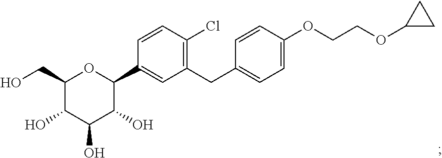 [SMILES: OC[C@H]1O[C@@H](c2ccc(Cl)c(Cc3ccc(OCCOC4CC4)cc3)c2)[C@H](O)[C@@H](O)[C@@H]1O]
(20) Janagliflozin, represented by the following formula: 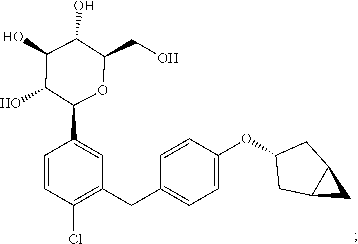 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]
(21) Rongliflozin; 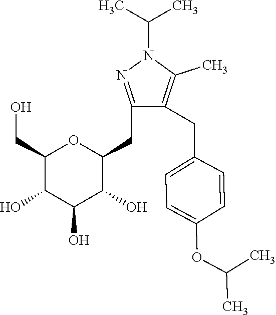 [SMILES: Cc1c(Cc2ccc(OC(C)C)cc2)c(C[C@@H]2O[C@H](CO)[C@@H](O)[C@H](O)[C@H]2O)nn1C(C)C]
(22) Wanpagliflozin;
(23) Enavogliflozin, represented by the following formula: 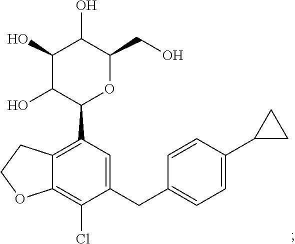 [SMILES: OC[C@H]1O[C@@H](c2cc(Cc3ccc(C4CC4)cc3)c(Cl)c3c2CCO3)C(O)[C@@H](O)C1O]
and
(24) TFC-039, represented by the following formula:

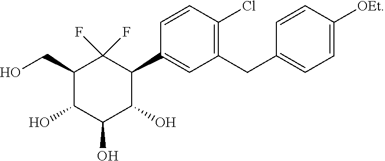 [SMILES: CCOc1ccc(Cc2cc([C@H]3[C@H](O)[C@@H](O)[C@H](O)[C@@H](CO)C3(F)F)ccc2Cl)cc1]

8. The system according to claim 7, wherein the concentration of the one or more SGLT-2 inhibitor compound(s) within the liquid pharmaceutical composition is from 0.1 mg/mL to 20 mg/mL.

9. The system according to claim 7, wherein the one or more SGLT-2 inhibitor compound(s) comprises Velagliflozin.

10. The system according to claim 7, wherein Velagliflozin is the only SGLT-2 inhibitor contained in the liquid pharmaceutical composition.

11. The system according to claim 10, wherein the concentration of Velagliflozin within the liquid pharmaceutical composition is 1.2 mg/mL or 15 mg/mL.

12. The system according to claim 7, wherein the liquid pharmaceutical composition includes the one or more SGLT-2 inhibitor compound(s) in solved or suspended form.

13. The system according to claim 6, wherein the liquid pharmaceutical composition includes one or more organic polar solvents selected from the group consisting of ethanol, propylene glycol, and glycerol.

14. The system according to claim 13, wherein the liquid pharmaceutical composition includes an organic polar solvent comprising propylene glycol and at least one of ethanol and glycerol.

15. The system according to claim 1, further comprising:
an adaptor connected with an opening in the container, the adaptor comprising a hollow connection member that facilitates coupling of the barrel forward end with the connection member to facilitate transfer of the liquid pharmaceutical composition to the first portion of the barrel, wherein a portion of the hollow connection member of the adaptor has an outer diameter that is substantially similar to an inner diameter at the opening in the container to establish a frictional and fluid tight connection between the adaptor and the container, and the hollow connection member of the adaptor has an inner diameter that is substantially similar to an outer diameter of the barrel forward end to establish a frictional and fluid tight connection between the adaptor and the barrel in response to insertion of the barrel forward end into the hollow connection member.

16. The system according to claim 15, wherein the adaptor is formed of a polymer material comprising, preferably consisting of, low density polyethylene, and the container is formed of a polymer material comprising, preferably consisting of, high density polyethylene.

17. A method of treatment and/or prevention of a metabolic disorder and/or another medical condition of a patient by administering a dosage of a liquid pharmaceutical composition comprising one or more SGLT-2 inhibitor compound(s) to the patient, the method comprising:
providing a syringe and a container that includes the liquid pharmaceutical composition, the syringe comprising a barrel and a plunger, wherein:
the barrel includes a hollow elongated member with an open barrel forward end and an open barrel rear end, a first portion that extends from the open barrel forward end to a transition section disposed along the hollow elongated member, the first portion having a first cross-sectional dimension, and a second portion that extends from the transition section to the open barrel rear end, the second portion having a second cross-sectional dimension that is greater than the first cross-sectional dimension;
the plunger includes an elongated member and a dosage scale, the elongated member including a plunger forward end and a plunger rear end, and the dosage scale comprises a plurality of marks disposed on a surface wall section of the plunger and aligned in a direction that corresponds with a lengthwise dimension of the plunger; and
the plunger and the barrel are dimensioned such that a portion of the plunger including the plunger forward end is insertable within the hollow elongated member of the barrel at the open barrel rear end, and the plunger is extendible within the barrel until the plunger forward end engages with an internal surface portion of the hollow elongated member at the barrel forward end; and
withdrawing a desired dosage amount of the liquid pharmaceutical composition from the container into the first portion of the barrel of the syringe by displacing the plunger forward end a selected distance from the barrel forward end until a selected mark of the plurality of marks for the dosage scale aligns with an exterior surface of the barrel at the barrel rear end.

18. The method according to claim 17, wherein the administering the desired dosage amount of the liquid pharmaceutical composition from the syringe to the patient comprises oral administration or parenteral administration to the patient, preferably oral administration, wherein more preferably the administering the desired dosage amount of the liquid pharmaceutical composition from the syringe to the patient comprises oral administration to the patient by direct transfer of the liquid pharmaceutical composition from the syringe orally to the patient or by indirect transfer of the liquid pharmaceutical composition from the syringe to food or liquid for consumption by the patient.

19. The method according to claim 17, wherein the desired dosage amount is no greater than 3 mL, or no greater than 2 mL, or no greater than 1 mL, or no greater than 0.9 mL, or no greater than 0.8 mL, or no greater than 0.7 mL, or no greater than 0.6 mL, or no greater than 0.5 mL, or no greater than 0.4 mL.

20. The method according to claim 17, wherein the at least one SGLT-2 inhibitor compound is selected from the group consisting of:
(1) a glucopyranosyl-substituted benzene derivative represented by the following formula: 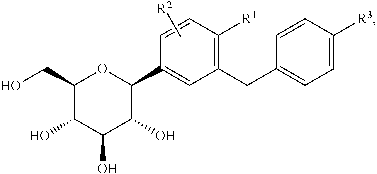
wherein R¹ denotes cyano, Cl or methyl (most preferably cyano),
R² denotes H, methyl, methoxy or hydroxy (most preferably H), and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by the following formula: 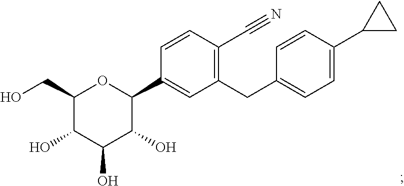 [SMILES: N#Cc1ccc([C@@H]2O[C@H](CO)[C@@H](O)[C@H](O)[C@H]2O)cc1Cc1ccc(C2CC2)cc1]
(3) Dapagliflozin, represented by the following formula: 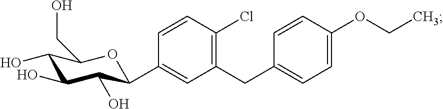
(4) Canagliflozin, represented by the following formula: 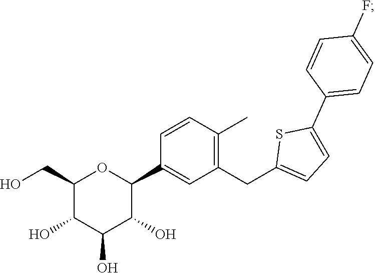 [SMILES: Cc1ccc([C@@H]2O[C@H](CO)[C@@H](O)[C@H](O)[C@H]2O)cc1Cc1ccc(-c2ccc(F)cc2)s1]
(5) Empagliflozin, represented by the following formula: 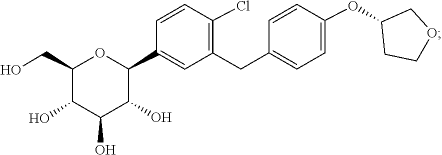 [SMILES: OC[C@H]1O[C@@H](c2ccc(Cl)c(Cc3ccc(O[C@H]4CCOC4)cc3)c2)[C@H](O)[C@@H](O)[C@@H]1O]
(6) Luscogliflozin, represented by the following formula: 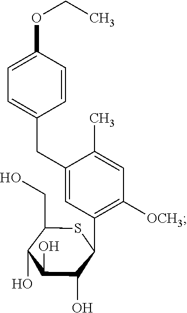
(7) Tofogliflozin, represented by the following formula: 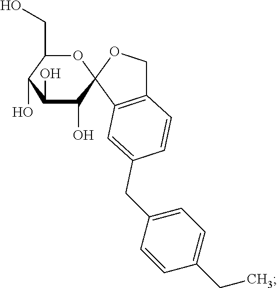
(8) Ipragliflozin, represented by the following formula: 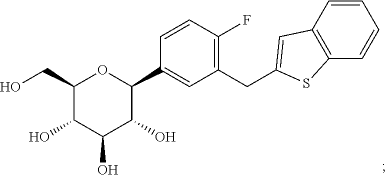 [SMILES: OC[C@H]1O[C@@H](c2ccc(F)c(Cc3cc4ccccc4s3)c2)[C@H](O)[C@@H](O)[C@@H]1O]
(9) Ertugliflozin, represented by the following formula: 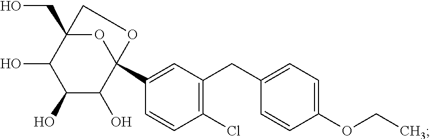 [SMILES: CCOc1ccc(Cc2cc([C@]34OC[C@](CO)(O3)C(O)[C@H](O)C4O)ccc2Cl)cc1]
(10) Atigliflozin, represented by the following formula: 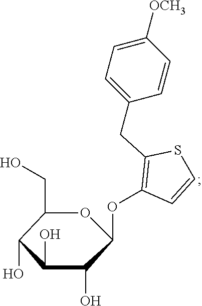
(11) Remogliflozin, represented by the following formula: 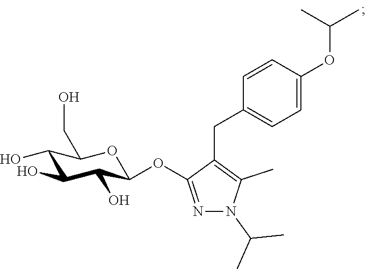
(11A) Remogliflozin etabonate, represented by the following formula: 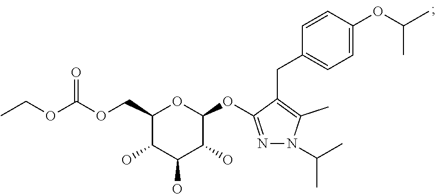 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]
(12) a thiophene derivative represented by the following formula: 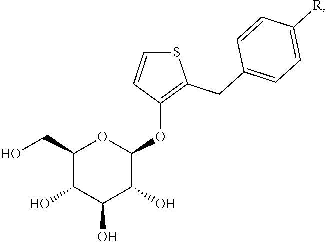
wherein R denotes methoxy or trifluoromethoxy;
(13) 1-(β-D-glucopyranosyl)-4-methyl-3-[5-(4-fluorophenyl)-2-thienylmethyl]benzene, represented by the following formula: 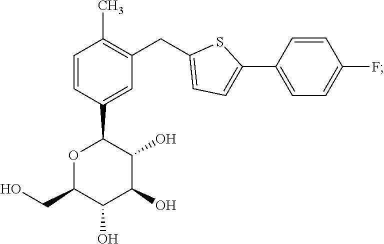 [SMILES: Cc1ccc([C@@H]2O[C@H](CO)[C@@H](O)[C@H](O)[C@H]2O)cc1Cc1ccc(-c2ccc(F)cc2)s1]
(14) a spiroketal derivative represented by the following formula: 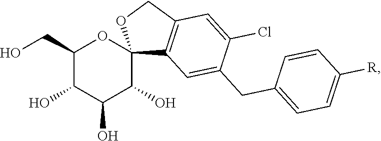
wherein R denotes methoxy, trifluoromethoxy, ethoxy, ethyl, isopropyl or tert. butyl;
(15) a pyrazole-O-glucoside derivative represented by the following formula: 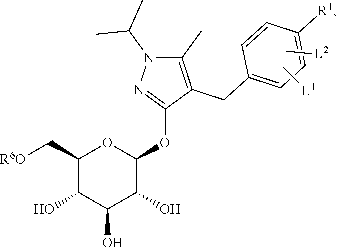
wherein:
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by the following formula: 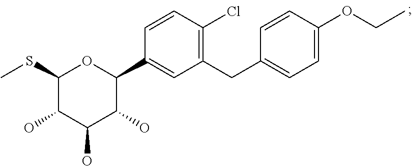 [SMILES: CCOc1ccc(Cc2cc([C@@H]3O[C@H](SC)[C@@H](O)[C@H](O)[C@H]3O)ccc2Cl)cc1]
(17) Sergliflozin, represented by the following formula: 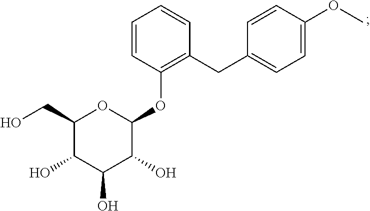 [SMILES: COc1ccc(Cc2ccccc2O[C@@H]2O[C@H](CO)[C@@H](O)[C@H](O)[C@H]2O)cc1]
(18) a compound represented by the following formula: 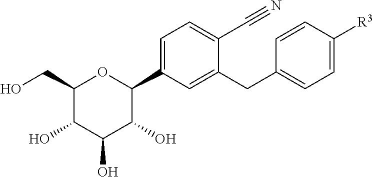
wherein:
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl, or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by the following formula: 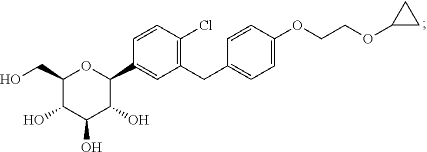 [SMILES: OC[C@H]1O[C@@H](c2ccc(Cl)c(Cc3ccc(OCCOC4CC4)cc3)c2)[C@H](O)[C@@H](O)[C@@H]1O]
(20) Janagliflozin, represented by the following formula: 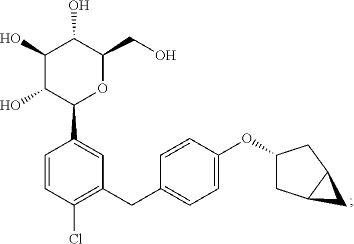 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]
(21) Rongliflozin: 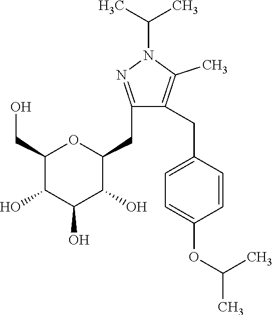 [SMILES: Cc1c(Cc2ccc(OC(C)C)cc2)c(C[C@@H]2O[C@H](CO)[C@@H](O)[C@H](O)[C@H]2O)nn1C(C)C]
(22) Wanpagliflozin;
(23) Enavogliflozin, represented by the following formula: 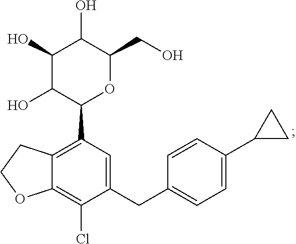 [SMILES: OC[C@H]1O[C@@H](c2cc(Cc3ccc(C4CC4)cc3)c(Cl)c3c2CCO3)C(O)[C@@H](O)C1O]
and
(24) TFC-039, represented by the following formula: 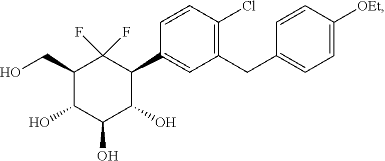 [SMILES: CCOc1ccc(Cc2cc([C@H]3[C@H](O)[C@@H](O)[C@H](O)[C@@H](CO)C3(F)F)ccc2Cl)cc1]
wherein preferably the one or more SGLT-2 inhibitor compound(s) comprises Velagliflozin, more preferably wherein Velagliflozin is the only SGLT-2 inhibitor compound administered.

21. The method according to claim 17, wherein the metabolic disorder and/or other medical condition is selected from the group consisting of:
(i) a metabolic disorder of an equine animal, wherein preferably the metabolic disorder is one or more disorders selected from insulin resistance, hyperinsulinemia, impaired glucose tolerance, dyslipidemia, dysadipokinemia, subclinical inflammation, systemic inflammation, low grade systemic inflammation, obesity, and/or regional adiposity, wherein preferably the metabolic disorder is insulin resistance, hyperinsulinemia, and/or a clinical condition associated with insulin resistance and/or hyperinsulinemia; wherein preferably said clinical condition is one or more conditions selected from impaired glucose tolerance, dyslipidemia, dysadipokinemia, subclinical inflammation, systemic inflammation, low grade systemic inflammation, obesity, and/or regional adiposity;
(ii) a metabolic disorder of an equine animal, wherein the metabolic disorder is one or more disorders selected from laminitis, vascular dysfunction, hypertension, hepatic lipidosis, atherosclerosis, hyperadrenocorticism, Pituitary Pars Intermedia Dysfunction and/or Equine Metabolic Syndrome, wherein preferably the metabolic disorder is a clinical condition/sign associated with insulin resistance and/or hyperinsulinemia, wherein said clinical condition/sign preferably is one or more conditions selected from laminitis, vascular dysfunction, hypertension, hepatic lipidosis, atherosclerosis, hyperadrenocorticism, Pituitary Pars Intermedia Dysfunction and/or Equine Metabolic Syndrome;
(iii) a metabolic disorder of a feline animal, wherein preferably the metabolic disorder is one or more selected from the group consisting of: ketoacidosis, pre-diabetes, diabetes mellitus type 1 or type 2, insulin resistance, acromegaly, diabetes with elevated IGF-1 concentration, obesity, hyperglycemia, impaired glucose tolerance, hyperinsulinemia, dyslipidemia, dysadipokinemia, subclinical inflammation, systemic inflammation, low grade systemic inflammation, hepatic lipidosis, atherosclerosis, inflammation of the pancreas, neuropathy and/or Syndrome X (metabolic syndrome) and/or loss of pancreatic beta cell function and/or wherein the remission of the metabolic disorder, preferably diabetic remission, is achieved and/or maintained;
(iv) a metabolic disorder of a canine animal, wherein preferably the metabolic disorder is one or more selected from the group consisting of: ketoacidosis, pre-diabetes, insulin dependent diabetes mellitus, insulin resistance diabetes, insulin resistance, obesity, hyperglycemia, hyperglycemia induced cataract formation, impaired glucose tolerance, hyperinsulinemia, dyslipidemia, dysadipokinemia, subclinical inflammation, systemic inflammation, low grade systemic inflammation, hepatic lipidosis, inflammation of the pancreas, metabolic disorder consequences, such as hypertension, renal dysfunction and/or musculoskeletal disorders, and/or Syndrome X (metabolic syndrome), preferably pre-diabetes, insulin dependent diabetes mellitus, insulin resistance diabetes, insulin resistance, wherein preferably the development of hyperglycemia induced cataract formation is prevented or remission is achieved and/or wherein preferably the development of metabolic disorder consequences, such as hypertension, renal dysfunction and/or musculoskeletal disorders, is prevented or progression is slowed or remission is achieved;
(v) a cardiac disease of a feline animal, wherein preferably the cardiac disease is one or more selected from the group consisting of: heart failure, heart failure due to one or more cardiomyopathies, heart failure due to hypertrophic cardiomyopathy (HCM), heart failure due to restrictive cardiomyopathy (RCM), heart failure due to dilated cardiomyopathy (DCM), heart failure due to unclassified cardiomyopathy (UCM), heart failure due to arrhythmogenic right ventricular cardiomyopathy (ARVC), hypertrophic cardiomyopathy (HCM), restrictive cardiomyopathy (RCM), dilated cardiomyopathy (DCM), unclassified cardiomyopathy (UCM), and/or arrhythmogenic right ventricular cardiomyopathy (ARVC); preferably selected from the group consisting of: heart failure due to one or more cardiomyopathies, heart failure due to hypertrophic cardiomyopathy (HCM), hypertrophic cardiomyopathy (HCM);
(vi) drying-off of a non-human mammal, preferably ruminant, wherein preferably the medical condition is one or more selected from the group consisting of: improving and/or facilitating the drying-off of a non-human mammal, preferably ruminant, reducing the milk production, preferably milk production and/or secretion, in a pregnant and/or lactating non-human mammal, preferably ruminant, decreasing milk accumulation and/or engorgement in the udder, preferably udder and/or mammary gland, of a non-human mammal, preferably ruminant, decreasing the discomfort associated with udder engorgement, such as increasing the daily lying time and/or reduction of stress, of a non-human mammal, preferably ruminant, decreasing milk leakage after drying-off of a non-human mammal, preferably ruminant, decreasing the incidence of intra-mammary infections (IMI), preferably mastitis and/or metritis, in a non-human mammal, preferably ruminant;
(vii) a cardiac disease of a non-human mammal, excluding a feline, in particular a canine, wherein preferably the cardiac disease is one or more selected from the group consisting of: heart failure; congestive heart failure; asymptomatic/preclinical/occult heart failure; heart failure due to (myxomatous) mitral valve disease [(M)MVD]; congestive heart failure due to (myxomatous) mitral valve disease [(M)MVD]; asymptomatic/preclinical/occult heart failure due to (myxomatous) mitral valve disease [(M)MVD]; (myxomatous) mitral valve disease [(M)MVD]; clinically overt (myxomatous) mitral valve disease [(M)MVD]; asymptomatic/preclinical/occult (myxomatous) mitral valve disease [(M)MVD]; heart failure due to dilated cardiomyopathy (DCM); congestive heart failure due to dilated cardiomyopathy (DCM); asymptomatic/preclinical/occult heart failure due to dilated cardiomyopathy (DCM); dilated cardiomyopathy (DCM); clinically overt dilated cardiomyopathy (DCM); asymptomatic/preclinical/occult dilated cardiomyopathy (DCM); aortic stenosis (valvular, supravalvular and/or subvalvular);
(viii) hypertension in a non-human mammal, preferably a carnivore, more preferably a cat or a dog, wherein preferably the hypertension is one or more selected from the group consisting of: situational hypertension, secondary hypertension and idiopathic hypertension, wherein preferably the secondary hypertension is selected from the group consisting of hypertension associated with chronic kidney disease (CKD), diabetes, obesity, heart disease, endocrine disease, such as Cushing's disease, hyperthyroidism, acromegaly, and elevated blood pressure (BP) induced by medicaments, preferably by glucocorticoids, mineralocorticoids, erythropoiesis-stimulating agents, ephedrine and/or high dose sodium chloride;
(ix) a renal disease of a non-human mammal, preferably a carnivore, more preferably a cat or a dog, wherein preferably the renal disease is one or more selected from the group consisting of: renal dysplasia, glomerulopathy, polycystic kidney disease, amyloidosis, tubulo-nephritis/tubulointerstitial nephritis (TIN), acute kidney disease, chronic kidney disease;
wherein preferably the dosage amount is from 0.01 to 10 mg/kg bodyweight of the patient.
